(19) United States
(12) Patent Application Publication
(10) Pub. No.: US 2015/0285818 A1
(43) Pub. Date: Oct. 8, 2015

(54) Switchable Reporter Enzymes for Homogenous Antibody Detection

(71) Applicant: TECHNISCHE UNIVERSITEIT EINDHOVEN, Eindhoven (NL)

(72) Inventors: Sambashiva Banala, Ashburn, VA (US); Stijn Johannes Amanda Aper, Zevenbergschen Hoek (NL); Maarten Merkx, Best (NL)

(21) Appl. No.: 14/430,530

(22) PCT Filed: Sep. 27, 2013
(86) PCT No.: PCT/EP2013/070248

§ 371 Date: Mar. 23, 2015

Related U.S. Application Data
(60) Provisional application No. 61/706,186, filed on Sep. 27, 2012.

(51) Int. Cl. G01N 33/68; G01N 33/58

(52) U.S. Cl. CPC G01N 33/6854; G01N 33/581

(57) ABSTRACT

A generic biosensor strategy was developed for the construction of switchable antibody reporter enzymes that allow direct detection of antibodies in solution including serum. The biosensor principle is based on the antibody-induced disruption of the intramolecular interaction between a reporter enzyme and its inhibitor and takes advantage of a unique structural property shared by all antibody classes, the presence of two identical antigen binding sites separated by a distance of approximately 100 Å. Unlike previous strategies, this biosensor design is intrinsically modular, allowing the construction of e.g. β-lactamase reporter enzymes for in principle any target antibody without cumbersome optimization/screening procedures. General guidelines are provided for the construction of reporter enzymes using enzyme-inhibitor pairs.

### FIELD OF THE INVENTION

This invention relates to detection of antibodies for the diagnosis of diseases, immunizations, immune responses, allergies, or the like.

### BACKGROUND OF THE INVENTION

Antibody detection is essential for the diagnosis of many disease states, including infectious diseases, autoimmune diseases and allergies. While a wide variety of analytical techniques have been developed for the detection of antibodies in blood, saliva and other bodily fluids, many of them come with intrinsic limitations such as the requirement for multiple time-consuming incubation steps (ELISA and other heterogeneous, sandwich-type assays), multiple reagents, and/or sophisticated equipment (e.g. surface plasmon resonance).

New generic antibody detection strategies in which molecular recognition and signal generation are integrated within a single protein would be ideal, in particular for high-throughput screening and point-of-care applications. From a protein engineering perspective, the key question is then how antibody binding can be translated into a readily detectable signal change.

One approach for homogenous antibody detection directly in solution is to make use of fluorescently labeled epitopes (or mimotopes) whose fluorescence properties such as intensity, polarization and lifetime are significantly changed upon antibody binding. A sophisticated example is the development of peptide molecular beacons in which peptide epitopes are flanked by two synthetic fluorophores. These probes adopt a rigid and extended conformation upon antibody binding which results in significant changes either in energy transfer between donor and acceptor or pyrene excimer fluorescence. Other FRET-based approaches take advantage of the presence of two identical antigen binding domains, either by inducing the interaction between donor- and acceptor-labeled peptide-oligonucleotides, or vice versa, by a binding-induced disruption of an intramolecular interaction between donor and acceptor fluorescent proteins.

Although these strategies should be generally applicable to detect a wide variety of antibodies, their sensitivity is fundamentally limited by the concentration of fluorescent probe that can be reliably detected. In other words, they lack the enzymatic amplification step that is characteristic of ELISA.

Several groups have explored strategies to couple antibody recognition directly to a change in enzymatic activity. A common approach is to introduce peptide epitopes at permissive sites within reporter enzymes such as β-galactosidase, β-lactamase, alkaline phosphatase and lysozyme. However, these hybrid enzymes are catalytically compromised and analyte binding often results in a further decrease in activity, which is a serious drawback from an application point of view. Even when an increase in activity is observed, allosteric enzymes typically display a relatively modest 2-5 fold change in activity, resulting in high background activities. Finally, since their performance depends on subtle allosteric mechanisms, development of these systems is very much a process of trial-and-error rather than rational design.

Combinatorial approaches such as phage display and in vivo selection strategies have been reported in an effort to make development of these allosterically regulated reporter enzymes more efficient, but these approaches have not solved the intrinsic problem of small changes in enzyme activity.

A different strategy that has been pursued with some success is to make use of antibody-induced oligomerization of reporter enzymes or complementation of split reporter enzymes. These approaches utilize the bivalent nature of antibodies to bring together two proteins (or protein-fragments) to form an active enzyme. Unlike the allosterically regulated enzymes, which need to be developed for each new antibody/epitope, these approaches should be applicable to any target antibody, but they also have some intrinsic limitations. For the split enzyme systems, the reconstituted enzyme activity is typically low (only 1-2%) compared to its parent enzyme. Furthermore, the fragments have a tendency to self-associate, which makes the sensor performance dependent on the sensor concentrations and therefore less robust than a single protein sensor.

The present invention provides a different design principle for the development of antibody-responsive reporter enzymes that addresses many of the limitations described supra.

### SUMMARY OF THE INVENTION

Detection of antibodies is essential for the diagnosis of many diseases including infectious diseases, autoimmune diseases and allergies. Current heterogeneous assays such as ELISA (enzyme-linked immune sorbent assay) require multiple time-consuming binding and washing steps, which limits their application in low cost point of care diagnostics and high-throughput screening.

With this invention, we present a new approach that allows one-step detection of antibodies directly in solution using a switchable reporter enzyme. The sensor design is highly modular, including the enzyme TEM1-β-lactamase (PDB Accession/Version No 1ZG4_A, GI:67464382) fused to its natural inhibitor protein (BLIP; UniProtKB/Swiss-Prot Accession/Version No P35804.1, GI:543897) via a long, semi-flexible peptide linker. Bivalent binding of antibody to two epitope sequences introduced at the ends of the linker disrupts the enzyme-inhibitor complex, resulting in an increase in enzyme activity that can be monitored using simple colorimetric or fluorescent read outs. Using the anti-HIV1 p¹⁷ antibody as an examplary target, the intramolecular affinity for the enzyme-inhibitor was optimized to yield a reporter enzyme whose activity increased 10-fold in the presence of pM concentrations of the target antibody (Kd=0.17 nM). A reporter enzyme that targets a completely different antibody could be obtained without any further sensor optimization by simply replacing the epitope sequence.

A thermodynamic scheme describing the dependence of sensor performance on the linker properties and the affinities of the antibody-epitope and enzyme-inhibitor pairs was developed and tested by deliberate attenuation of the antibody-epitope interaction. Unlike previous protein engineering approaches based on allosteric modulation of an enzyme active site, our approach provides a generic, modular framework for the rational design of antibody reporter enzymes for homogenous immunoassays.

In one embodiment the invention is an antibody detection method where a biosensor is used for detecting an antibody. The biosensor is an enzyme covalently linked to an inhibitor protein via a peptide linker having two epitopes at the ends of the peptide linker. The biosensor is defined by two equilibrium constants, K_{closed-open,1} and K_{closed-open,2} that describe the equilibrium between a closed and an open state of the biosensor in the absence and presence, respectively, of the antibody according to:

K_{closed-open,1}=K_{d(EI)}/C_{eff,(EI)}, and

K_{closed-open,2}=0.5*K_{d,(EI)}/K_{d(AP)}*C_{eff,(AP)}/C_{eff(EI)}
whereby:
K_{d(AP)} is an intermolecular dissociation constant of a monovalent binding of the antibody and the epitope,
K_{d(EI)} is an intermolecular dissociation constant of the binding of the enzyme and the inhibitor protein,
C_{eff(EI)} is an effective concentration of the inhibitor protein in proximity of the enzyme, and
C_{eff(AP)} is an effective concentration of a free epitope in proximity of the remaining antigen-binding domain of the antibody.

For the biosensor to work in detecting an antibody, K_{closed-open,1} is smaller than K_{closed-open,2}. In one example, K_{closed-open,1} is less than 3. In a preferred embodiment, K_{closed-open,1} is larger than 0 and less than 0.2. Regarding K_{closed-open,2}, in one example K_{closed-open,2} is greater than 0.2. In another example, K_{closed-open,2} is larger than 0.2 and less than 10⁶.

The equilibrium constants, K_{closed-open,1} and K_{closed-open,2}, can be determined by measuring the enzymatic activity of the biosensor in the absence (i.e. K_{closed-open,1}) and presence of saturating amount of target antibody (i.e. K_{closed-open,2}) and by comparing the enzymatic activity to that of the same concentration of the enzyme alone.

The K_{d(AP)} can be determined by titration of the antibody to its epitope peptide conjugated to a fluorescent group and monitoring the formation of the antibody-peptide complex by using fluorescence anisotropy. Alternatively, formation of the antibody-peptide complex can be monitored using surface plasmon resonance or isothermal titration calorimetry.

The K_{d(EI)} can be determined by measuring enzymatic activity of the enzyme as a function of substrate concentration (Michaelis Menten kinetics) in the absence and presence of a known concentration of the inhibitor domain. The Kᵢ(=K_{d(EI)}) was calculated using K_{M(+BLIP)}=K_{M}(1+([BLIP]/Kᵢ)), which represent the relation between the 2 K_{M} values, the inhibitor concentration and Kᵢ for a competitive inhibitor. Alternative methods for determining this constant include biophysical methods such as the use of surface plasmon resonance or isothermal titration calorimetry to monitor complex formation between enzyme and inhibitor.

C_{eff(EI)} can be derived after determining K_{d, EI} and K_{closed-open,1} from

K_{closed-open,1}=K_{d(EI)})/C_{eff,(EI)}.

C_{eff(AP)} can be derived after determining K_{d, EI}, Kd_{, AP}, C_{eff(EI)} and K_{closed-open,2}

K_{closed-open,2}=0.5*K_{d,(EI)})/K_{d(AP)}*C_{eff,(AP)}/C_{eff(EI)}.

In another embodiment the invention, an in vitro antibody-detecting method is provided. The method entails contacting a sample with a biosensor. The biosensor includes a reporter enzyme (for example, but not limited to, beta-lactamase), an inhibitor domain (for example, but not limited to, beta-lactamase inhibitor protein, BLIP) having affinity for the reporter enzyme, at least two epitopes, whereby each epitope has affinity for the antibody, and a linker. The method further entails determining the activity of the reporter enzyme in the presence of a sample, and attributing the activity of the reporter enzyme in the presence of the sample to the quantitative or qualitative presence or absence of an antibody. Now in the absence of the antibody, the biosensor is in a closed, inactive state in which at least some (e.g. at least 30%, 50% or 80% in different examples) of the reporter enzyme forms an intramolecular complex with the inhibitor domain. In different words, the equilibrium is to the left. A bivalent binding between two antigen binding domains present in the antibody and the (at least) two epitopes present at the ends of the linker between the reporter enzyme and the inhibitor domain in the biosensor changes the equilibrium between the closed (inactive) and open (active) state of the biosensor such that the amount of the reporter enzyme that forms an intramolecular complex with the inhibitor domain is decreased.

Also described is an in vitro antibody-detecting method. In this method, a sample is contacted with a biosensor which is displaceable between an open state and a closed state.

The biosensor includes a reporter enzyme (for example, but not limited to, beta-lactamase) or a fragment thereof, an inhibitor domain (for example, but not limited to, beta-lactamase inhibitor protein, BLIP) or a fragment thereof having affinity for the reporter enzyme, at least two epitopes, wherein each epitope has affinity for the antibody, and a linker separating the at least two epitopes. This method further entails determining the activity of the reporter enzyme in the presence of the sample, and attributing the activity of the reporter enzyme in the presence of the sample to the quantitative or qualitative presence or absence of the antibody. Now, in the closed state, the reporter enzyme and the inhibitor domain form an (inactive) intramolecular complex. The binding (e.g. bivalent binding) of an antibody to the at least two epitopes changes the equilibrium between the closed (inactive) and an open (active) state of the biosensor, which thereby displaces the biosensor from the closed state to the open state, such that the amount of reporter enzyme forming an intramolecular complex with the inhibitor domain is decreased.

Also described is a biosensor displaceable between an open state and a closed state. The biosensor includes a reporter enzyme (for example, but not limited to, beta-lactamase) or a fragment thereof, an inhibitor domain (for example, but not limited to, beta-lactamase inhibitor protein, BLIP) or a fragment thereof having affinity for the reporter enzyme, at least two epitopes, whereby each epitope has affinity for the antibody, and a linker separating the at least two epitopes.

In the closed state of this method, the reporter enzyme and the inhibitor domain form an (inactive) intramolecular complex. Further in this method, the binding of an antibody to the at least two epitopes changes the equilibrium between the closed (inactive) and an open (active) state of the biosensor, thereby displacing the biosensor from the closed state to the open state, such that the amount of said reporter enzyme forming an intramolecular complex with the inhibitor domain is decreased.

Optionally, binding of an antibody to the at least two epitopes changes the equilibrium between the closed (inactive) and an open (active) state of the biosensor, which thereby displaces the biosensor from the closed state to the open state, such that the activity of said reporter enzyme is increased. Optionally, in the presence of an antibody, the biosensor is in the open state. Further optionally, in the presence of an antibody, a bivalent binding between two antigen binding domains present in said antibody and the at least two epitopes displaces the biosensor to the open state. Still further optionally, in the presence of an antibody, a bivalent binding between two antigen binding domains present in the antibody and the at least two epitopes present at the ends of the linker between the reporter enzyme and the inhibitor domain in the biosensor, displaces the biosensor to the open state. Still further optionally, in the open state, the reporter enzyme is spaced apart from the inhibitor domain, thereby allowing activity of the reporter enzyme.

Optionally, in the absence of an antibody, the biosensor is in the closed state. Further optionally, in the absence of an antibody, the biosensor is in the closed state whereby at least some of the reporter enzyme forms an intramolecular complex with the inhibitor domain. Still further optionally, in the closed state, the reporter enzyme and the inhibitor domain form a molecular complex, thereby inhibiting activity of the reporter enzyme.

Optionally, the activity of the reporter enzyme is proportional to the quantitative or qualitative presence or absence of the antibody.

Optionally, the reporter enzyme is a polypeptide capable of catalysing the reaction of a non-visible substrate to a visible product. Optionally, the reporter enzyme is beta-lactamase or a fragment thereof. Further optionally, the reporter enzyme is TEM1 β-lactamase or a fragment thereof.

Optionally, the inhibitor domain is a polypeptide having affinity for the reporter enzyme and capable of forming a molecular complex with the reporter enzyme to inhibit activity of the reporter enzyme. Optionally, the inhibitor domain is beta-lactamase inhibitory protein (BLIP) or a fragment thereof. Alternatively, the inhibitor domain is an inhibitory peptide derived from phage display.

Optionally, the at least two epitopes are polypeptides or polypeptide fragments having affinity for the antibody and capable of binding, optionally selectively binding, to the antibody, optionally to the antigen-binding fragments of the antibody. Optionally, each epitope is capable of independently binding, optionally independently selectively binding, to the each respective antigen-binding fragment of the antibody.

Optionally, the linker is a polypeptide or polypeptide fragment including at least one flexible block of (GSG)₆. Further optionally, the linker further includes at least one α-helical block. Still further optionally, the linker further includes at least one α-helical block having six EAAAK repeats. Still further optionally, the linker further includes two α-helical blocks, each block having six EAAAK repeats.

Optionally, each epitope is located at each respective end of the linker. Further optionally, each of the reporter enzyme and the inhibitor domain is located at each respective end of the linker. Still further optionally, a first epitope and the reporter enzyme are located at a first end of the linker, and a second epitope and the inhibitor domain is located at a second, opposing end of the linker.

### BRIEF DESCRIPTION OF THE DRAWINGS

FIG. 1 shows according to an exemplary embodiment of the invention a switchable antibody reporter enzyme based on the antibody-induced disruption of an intramolecular complex between enzyme and inhibitor domains. (E=enzyme; I=inhibitor; S=substrate; P=product).

FIGS. 2A-C show according to an exemplary embodiment of the invention: (2A) shows a general structure of the antibody reporter enzymes. Abs-1-3 (SEQ ID NO: 12, 13, and 21 respectively) contain epitopes targeting the anti-HIV1-p17 antibody, Abs-4 (SEQ ID NO: 40) contains an HA-tag for binding anti-HA-tag antibodies.

(2B) shows a comparison of the β-lactamase activity of β-lactamase alone, Abs-1 (SEQ ID NO: 12), and Abs-2 (SEQ ID NO: 13) in the absence and presence of 200 nM anti-HIV1-p17 antibody.

(2C) shows a quantification of the data shown in FIG. 2B. Activity assays were done using 0.3 nM enzyme with 50 μM nitrocefin in 50 mM phosphate buffer (pH 7.0) containing 100 mM NaCl and 1 mg/mL BSA.

FIG. 3 shows according to an exemplary embodiment of the invention enzymatic activities of Abs-3 variants (SEQ ID NOs: 26-35) (0.1 nM) combining the E104D mutation in the -lactamase with various mutations in BLIP in the absence (white bars) and presence (dashed bars) of saturating amount (100 nM) of anti-HIV1-p17 antibody. All assays were done using 50 μM nitrocefin in 50 mM phosphate buffer (pH 7.0) containing 100 mM NaCl and 1 mg/mL BSA.

FIGS. 4A-C show according to an exemplary embodiment of the invention enzymatic activities of Abs3-1 (SEQ ID NO: 22) (4A) and Abs3-2 (SEQ ID NO: 37) (4B) as a function of anti-HIV1-p17 antibody concentration. FIG. 4C shows Abs3-2 (SEQ ID NO: 37) response with anti-HIV1-p¹⁷ (10 nM) alone, together with 0.1 mg/mL IgG mix (660 nM) and only with IgG mix (660 nM). Abs3-2 single epitope variants response towards the anti-HIV1-p17. All assays were done using 50 μM nitrocefin in 50 mM phosphate buffer (pH 7.0) containing 100 mM NaCl and 1 mg/mL BSA. In all the assays sensor concentration was 0.1 nM. (Ab=antibody).

FIG. 5 shows according to an exemplary embodiment of the invention enzymatic activity of Abs-4 (SEQ ID NO: 40) (0.1 nM) with different anti-HA antibody concentration. From the data a K_{d} value (0.20 nM) was obtained. Response towards the non-specific antibodies (IgG mix) is also shown. All assays were done using 50 μM nitrocefin in 50 mM phosphate buffer (pH 7.0) containing 100 mM NaCl and 1 mg/mL BSA.

FIG. 6 shows according to an exemplary embodiment of the invention Abs3-1 (SEQ ID NO: 22) response with saturating amount of anti-HIV1-p17 in buffer and serum with both nitrocefin and fluorescent substrates (CCF2-FA).

(6A) shows an activity assay with nitrocefin (50 μM) in buffer.

(6B) shows an assay with 1 μM CCF2-FA in buffer. In both cases 0.1 nM of the Abs3-1 (SEQ ID NO: 22) was used.

(6C) shows Abs3-1 (SEQ ID NO: 22) (5 nM) response in 10% FBS with 10 μM CCF2-FA in the presence and absence of the antibody.

(6D) shows a quantification of the activities in 6A, 6B and 6C. The maximum activity in the presence of antibody was taken as 100%.

FIG. 7 shows a thermodynamic model according to an exemplary embodiment of the invention. The thermodynamic model describes the bivalent binding between reporter enzyme and antibody in 3 steps. In step 1, antibody binds to one of the two epitopes in a intermolecular fashion (K_{d, AP}=intermolecular dissociation constant of the monovalent binding of antibody and epitope). In step 2, dissociation of the enzyme-inhibitor complex occurs. K_{d, EI}=intermolecular dissociation constant of the binding of enzyme and inhibitor, C_{eff, EI}=effective concentration of inhibitor in proximity of the enzyme. In step 3, antibody binds to second epitope of the sensor. C_{eff, AP}=effective concentration of free epitope in proximity of the remaining antigen binding domain of the antibody. Equations (1)-(4) are shown in FIG. 7.

FIG. 8 shows according to an exemplary embodiment of the invention activity change of Abs2-2 (SEQ ID NO: 20) (0.3 nM) as a function of anti-HIV1-p17 concentration. From the data a Kd value (108 nM) was obtained. Abs3-2 (SEQ ID NO: 37) (0.1 nM) activity change with the antibody concentration was also shown for comparison.

FIG. 9 shows according to an exemplary embodiment of the invention enzymatic activities of Abs-5 (SEQ ID NO: 42) as a function of antibody concentration. The solid lines represent a fit to Eq. 2, yielding a K_{d} value of 1.13 nM±0.46.

FIG. 10 shows according to an exemplary embodiment of the invention the affinity of the short HIV1-p17 epitope (WEKIRLR; (SEQ ID NO: 1).

FIGS. 11-12 show fluorescence polarization assays according to exemplary embodiments of the invention. FIG. 11 shows titration of 10 nM ELDRWEKIRLRP-GGG-C(fluorescein) with HIV1-p17 antibody monitored using fluorescence polarization. The data were fit to Eq. 8 yielding an apparent K_{d}=24±3 nM. FIG. 12 shows a competition assay with unlabelled peptide. Complex of antibody and fluorescein-labeled peptide (100 nM each) was titrated with non-labeled peptide (ELDRWEKIRLRP; (SEQ ID NO: 3). Fitting the data to Eqs. 9 and 10 yielded a K_{d} value of 42±1 nM.

FIGS. 13-14 show fluorescence polarization assays according to exemplary embodiments of the invention. FIG. 13 shows titration of 2 nM YPYDVPDYA-GGG-C(fluorescein) with anti-HA antibody monitored using fluorescence polarization. The data were fit to Eq. 8 yielding a K_{d}=0.58±0.22 nM. FIG. 14 shows a competition assay with unlabeled peptide. Complex of antibody and fluorescein-labeled peptide (10 nM each) was titrated with non-labeled peptide (YPYDVPDYA; (SEQ ID NO: 5). Fitting the data to Eqs. 9 and 10 yielded a K_{d} value of 4.5±1 nM.

FIG. 15 shows according to exemplary embodiments of the invention titration of 10 nM (EHKYSWKS-GGG-C(fluorescein)) with anti-Dengue-1 antibody monitored using fluorescence polarization. The data were fit to Eq. 8 yielding a K_{d} of 70±13 nM.

FIG. 16 shows according to exemplary embodiments of the invention enzymatic activity of Abs-3 mutants (0.3 nM) in the presence and absence of 200 nM of the anti-HIV1-p17 antibody. Antibody and sensor were incubated for 1 h at RT. Then 50 μM of nitrocefin was added and the measurement was started immediately. The assay was performed in pH 7.0 phosphate buffer (50 mM) that contains NaCl (100 mM) and BSA (1 mg/mL).

FIGS. 17-18 show Michaelis-Menten plots showing the activity of beta-lactamase-E104D (0.1 nM) as function of nitrocefin concentration in the absence and presence of 5 μM BLIP-E31A (SEQ ID NO: 47; FIG. 17), or 5 μM BLIP-F142A (SEQ ID NO: 48; FIG. 18). The solid lines represent fits to the Michaelis-Menten equation: FIG. 16: K_{M}=62 μM and K_{M} (+BLIP-E31A)=210 μM; Ki=2.1 μM. FIG. 17: K_{M}=88 μM and K_{M} (+BLIP-F142A)=239 μM; Ki=2.9 μM.

FIG. 19 show the primers for the mutagenesis according to an exemplary embodiment of the invention.

FIG. 20 show the primers used for generation of Abs-4 (SEQ ID NO: 40) and Abs-5 (SEQ ID NO: 42) according to an exemplary embodiment of the invention.

FIG. 21 show enzymatic activity of Abs-2 mutants (0.3 nM) in the presence and absence of 200 nM of the target antibody according to an exemplary embodiment of the invention.

### DETAILED DESCRIPTION

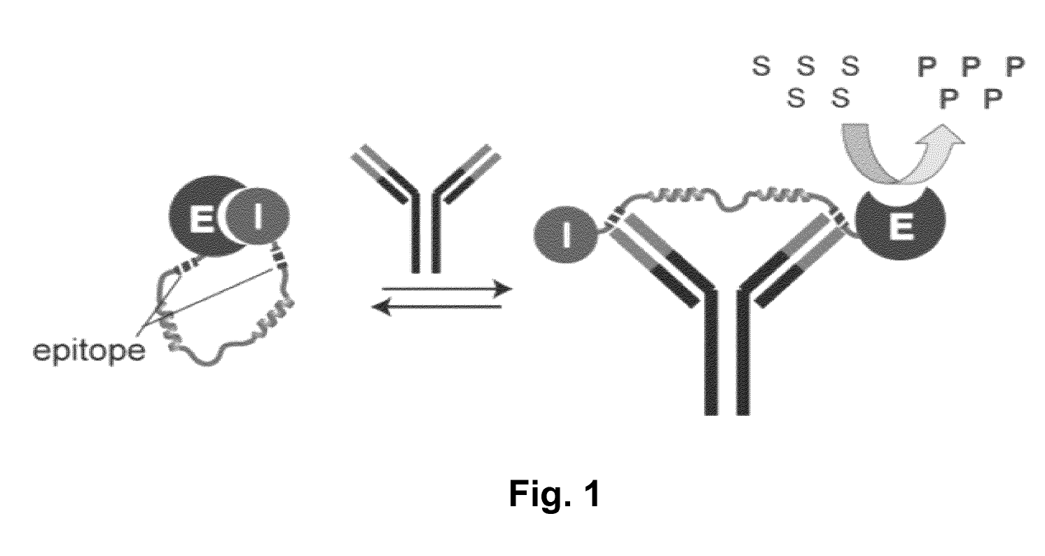

The switchable reporter enzymes can have a full length reporter enzyme that is conjugated to an inhibitor domain via a long semi-flexible linker, forming a catalytically inactive enzyme-inhibitor complex in the absence of its target antibody (FIG. 1). Peptide epitopes specific to the antibody of interest are introduced in the linker, one is next to the enzyme and the other adjacent to the inhibitor. Binding of a single antibody to both epitopes separates the enzyme-inhibitor complex, resulting in an increase in enzyme activity.

The feasibility of the technology of this invention was demonstrated using β-lactamase as a reporter enzyme. The affinity between this β-lactamase and its inhibitor protein BLIP was designed to yield single-protein reporter enzymes that allow detection of pM concentrations of specific antibodies using simple colorimetric or fluorescent read-outs. Moreover, because of the modular architecture of theses sensors, we show that epitope sequences can be readily exchanged without compromising the sensors' performance.

### Sensor Design

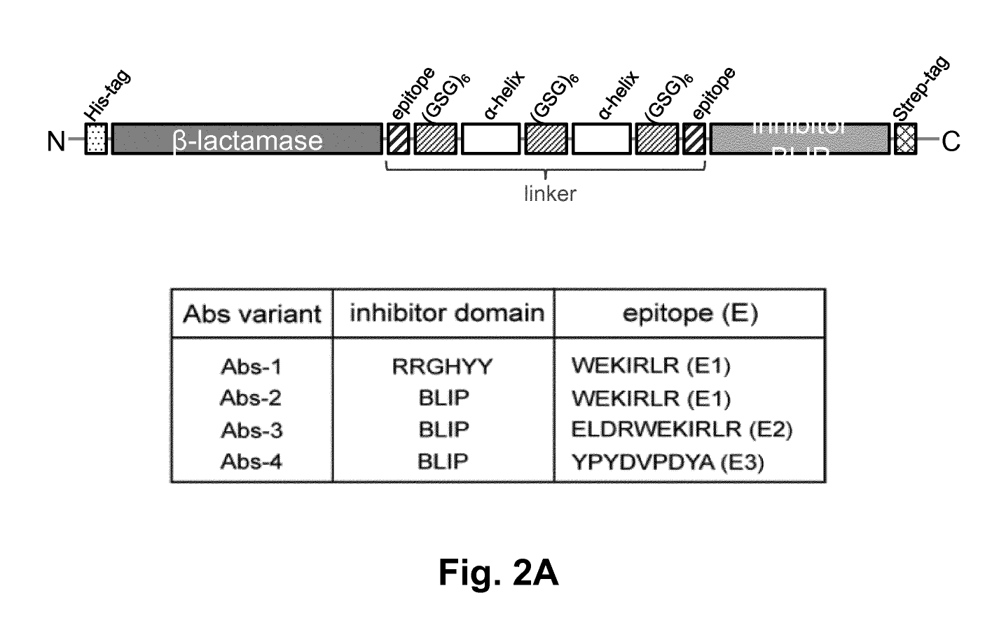

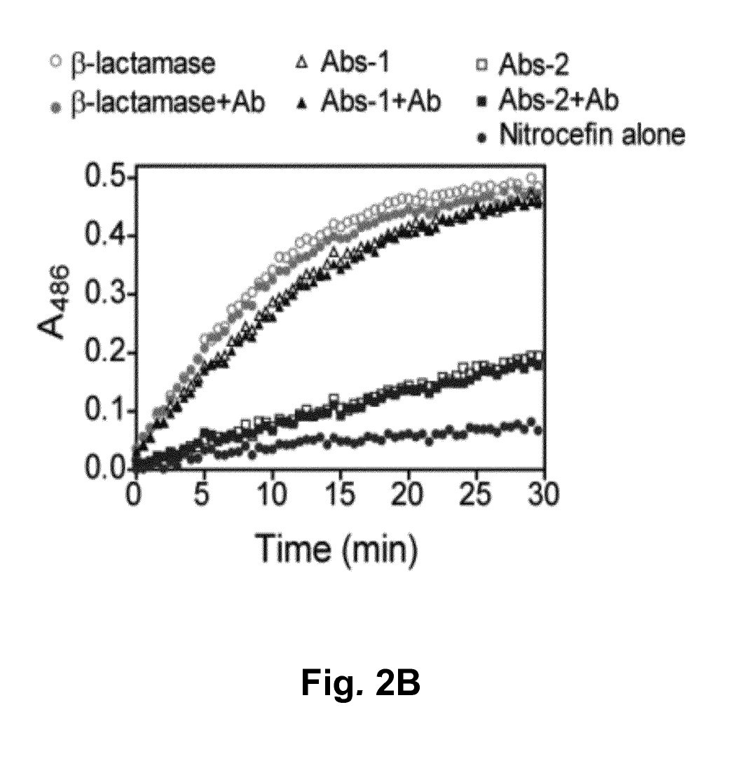

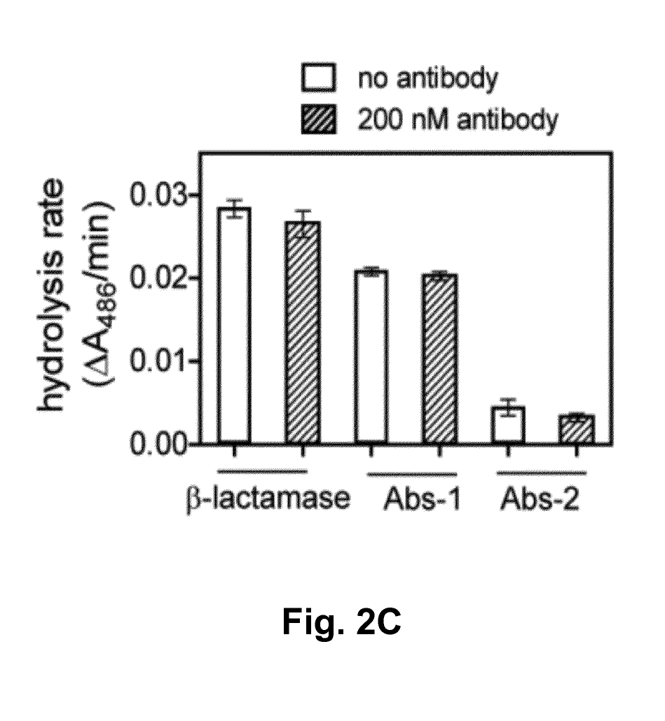

FIG. 2A shows the general design of our antibody-reporter enzymes. In this example, TEM1 β-lactamase was chosen as a reporter enzyme because it does not require oligomerization for activity and many substrates are available both for colorimetric and fluorescence detection. The enzyme has been a popular target for testing new protein engineering concepts, including sensors for antibody detection based on subtle allosteric regulation of enzyme activity. Most importantly, a variety of inhibitors are available for TEM1 β-lactamase, ranging from relatively weak inhibitory peptides derived from phage display (Kᵢ˜140 μM) to the natural β-lactamase inhibitor protein (BLIP; (SEQ ID NO:46), which has a Kᵢ of 0.5 nM.

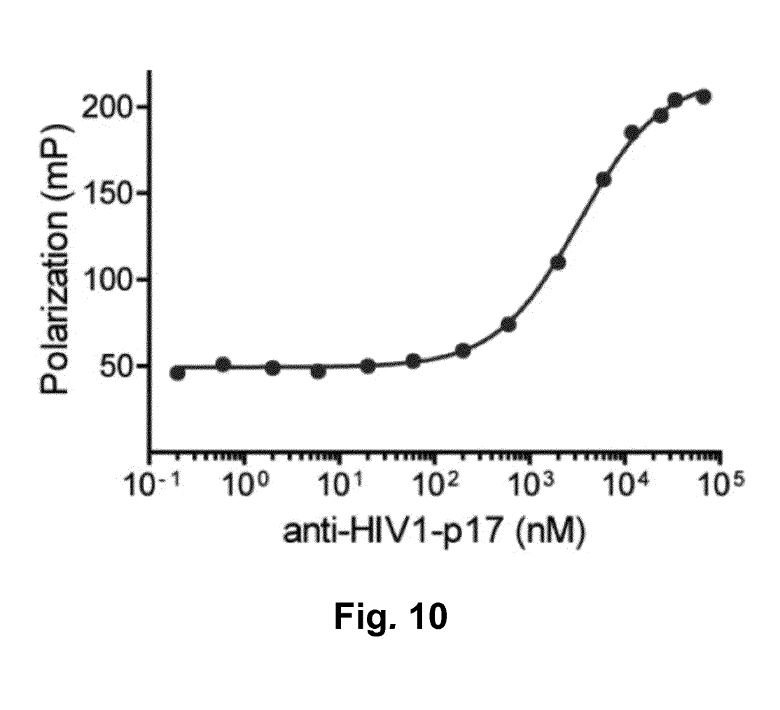

In an exemplary design we focused on developing a sensor for the detection of the anti-HIV1-p17 antibody. Several well-characterized linear epitope sequences are available for this antibody, which has made it a popular choice for the development of new homogeneous antibody detection assays. The linker between the enzyme and the inhibitor modules initially has two short peptide epitopes (WEKIRLR; (SEQ ID NO: 1), Kd˜3 μM; FIG. 10) specific for the HIV1-p17-antibody that are separated by three flexible blocks of (GSG)₆ and two α-helical blocks each having six EAAAK (SEQ ID NO: 7) repeats. This linker was also used in a recently reported FRET sensor protein based on the same switching strategy (Golynskiy, M. V., Rurup, W. F., and Merkx, M. (2010) Antibody detection by using a FRET-based protein conformational switch, ChemBioChem 11, 2264-2267). This study showed that introduction of two 45 Å-helical blocks in the flexible linker was essential for the linker to efficiently bridge the distance between the two antigen binding sites.

To establish the influence of inhibitor affinity, two variants were constructed in an exemplary embodiment containing either the weak-binding RRGHYY (SEQ ID NO: 8) peptide (Abs-1; SEQ ID NO: 12) or the strong BLIP protein as inhibitor domain (Abs-2; (SEQ ID NO: 13). To allow proper folding, proteins were expressed in E. coli BL21 (DE3) using a periplasmic leader sequence and purified using an N-terminal His-tag and a C-terminal Strep-tag. This two-step purification protocol ensures the isolation of full length protein only, without truncated version of the sensor lacking e,g. the inhibitor domain.

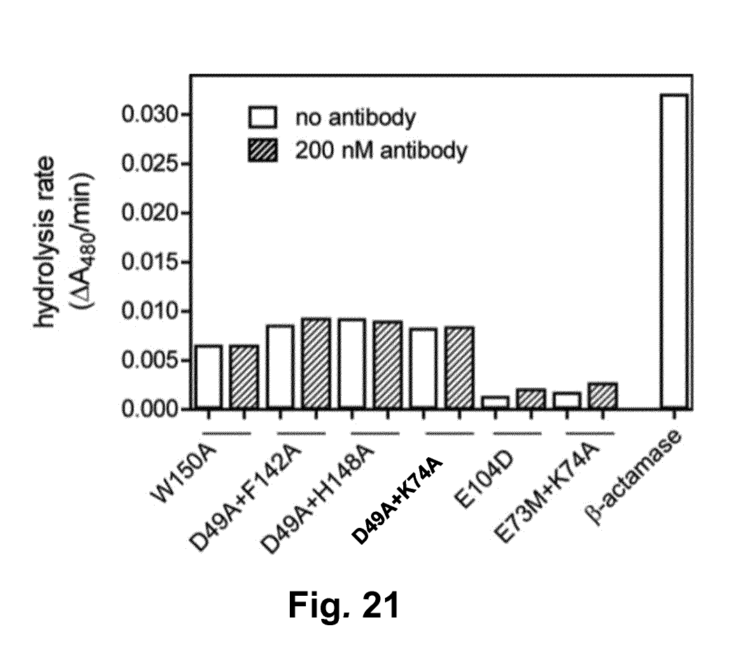

Enzymatic activity assays using the colorimetric substrate nitrocefin showed that the activity of Abs-1 (SEQ ID NO: 12) was similar to that of TEM1 β-lactamase in the absence of any inhibitor (FIGS. 2B-C). Moreover, no increase in enzymatic activity was observed upon addition of 200 nM anti-HIV1-p17. These results show that the affinity of the peptide inhibitor used in Abs-1 (SEQ ID NO: 12) is too weak to result in substantial enzyme inhibition in the absence of antibody. In contrast, the enzymatic activity of Abs-2 (SEQ ID NO: 13) was strongly inhibited compared to that of TEM1 β-lactamase, but no increase in activity was observed upon addition of 200 nM of the target antibody (FIGS. 2B-C). Introduction of several single and double point mutations in BLIP also did not show any response to the target antibody (FIG. 21). This result suggests that the intramolecular interaction between wild-type BLIP (and its mutants) and β-lactamase domains is actually too strong and that formation of a second epitope-antigen binding domain interaction is not sufficient to overcome this interaction.

### Design of Intramolecular Enzyme-Inhibitor Interaction

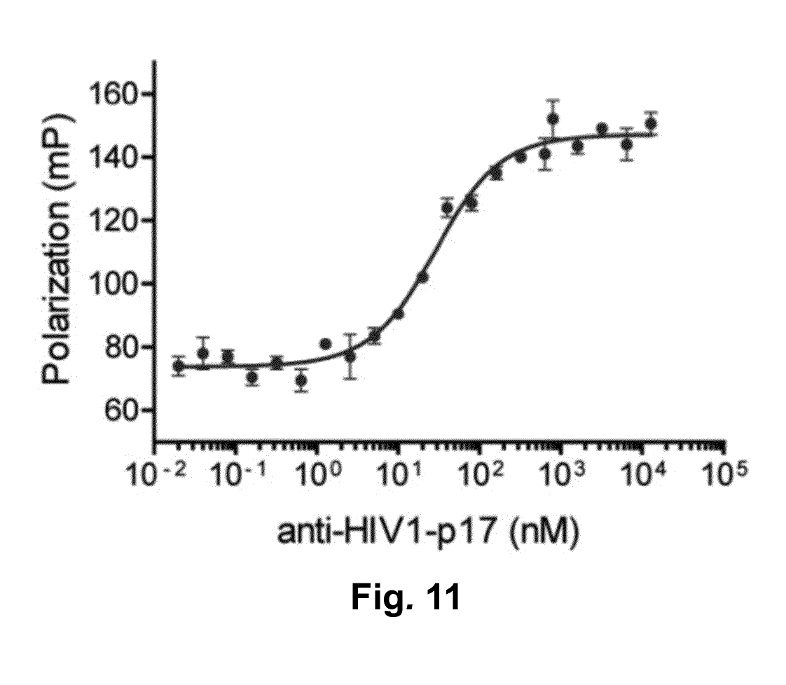

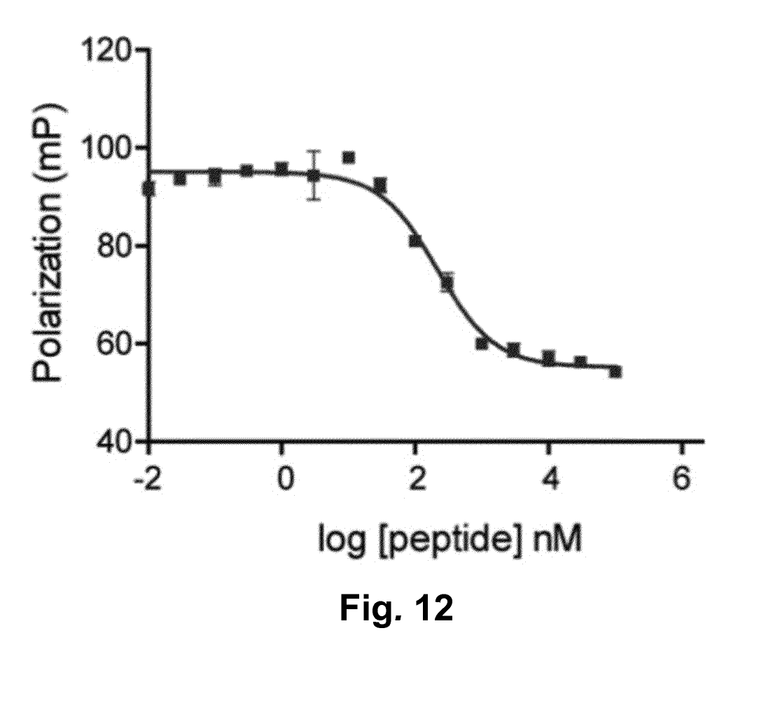

To provide a stronger driving force for disrupting the enzyme-inhibitor interaction, Abs-3 was created in which the short WEKIRLR (SEQ ID NO: 1) epitope was extended to a longer epitope (ELDRWEKIRLRP; SEQ ID NO: 3; K_{d}=42 nM; FIGS. 11-12). In addition, to systematically attenuate the interaction between the β-lactamase and BLIP, a series of Abs-3 variants was explored with mutations in either BLIP, β-lactamase, or both. The X-ray structure of the TEM1 β-lactamase-BLIP complex has been reported, and for many of the residues at the binding interface their contribution to the binding strength has been determined (see (A) Zhang, Z., and Palzkill, T. (2003) Determinants of binding affinity and specificity for the interaction of TEM-1 and SME-1 beta-lactamase with beta-lactamase inhibitory protein, The Journal of biological chemistry 278, 45706-45712, and (B) Natalie C. J. Strynadka, Susan E. Jensen, Pedro M. Alzari & Michael N. G. James (1996) A potent new mode of beta-lactamase inhibition revealed by the TEM-1/BLIP complex at 1.7 A resolution. Nature Structural & Molecular Biology 3, 290-297).

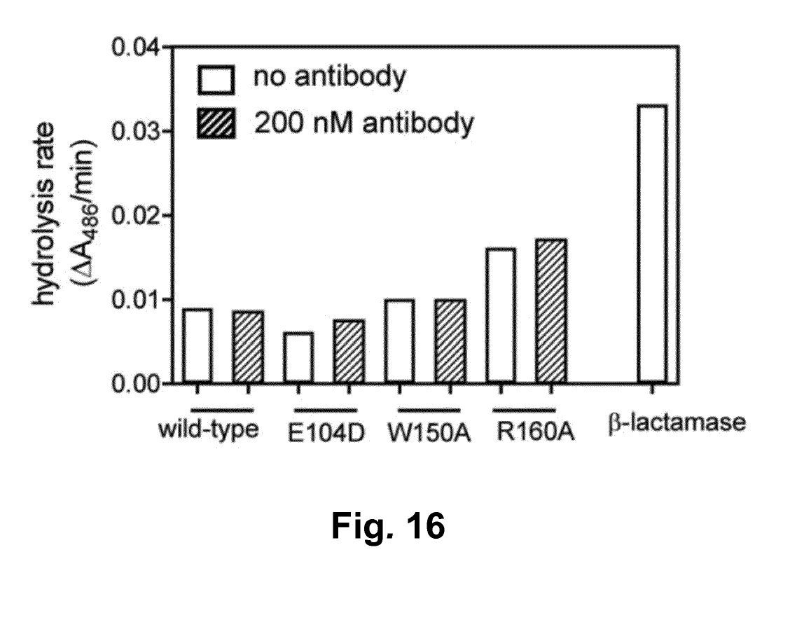

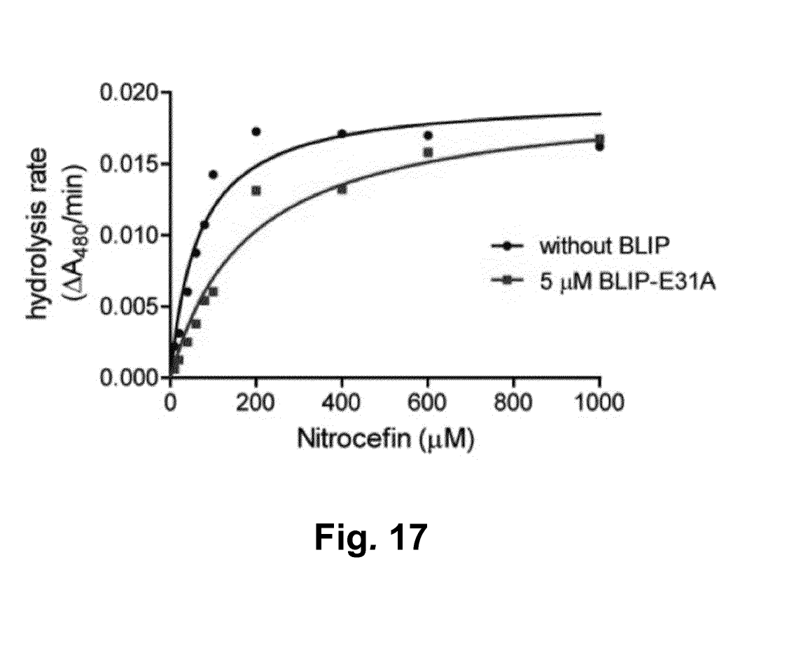

First, several single point mutations were introduced in BLIP. These mutations were previously reported to have affinities ranging from 20-150 nM, but none of them yielded antibody-responsive Abs-3 variants (FIG. 16). A similar result was obtained for a single point mutation in β-lactamase (E104D; SEQ ID NO: 45), which in a recent study was reported to have a 3 orders of magnitude lower affinity for BLIP than wt β-lactamase (K=1500 nM) (Hanes, M. S., Reynolds, K. A., McNamara, C., Ghosh, P., Bonomo, R. A., Kirsch, J. F., and Handel, T. M. (2011) Specificity and cooperativity at beta-lactamase position 104 in TEM-1/BLIP and SHV-1/BLIP interactions, Proteins 79, 1267-1276).

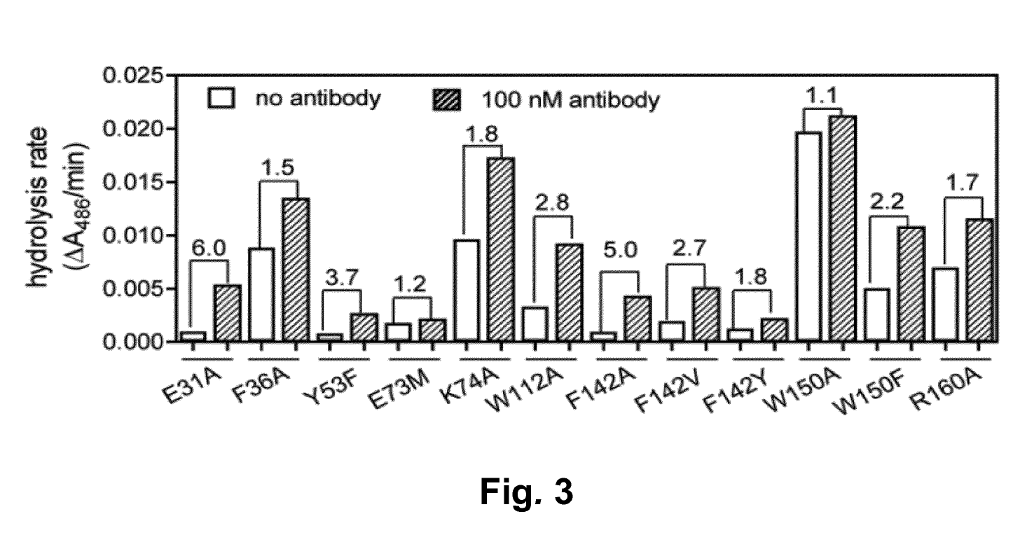

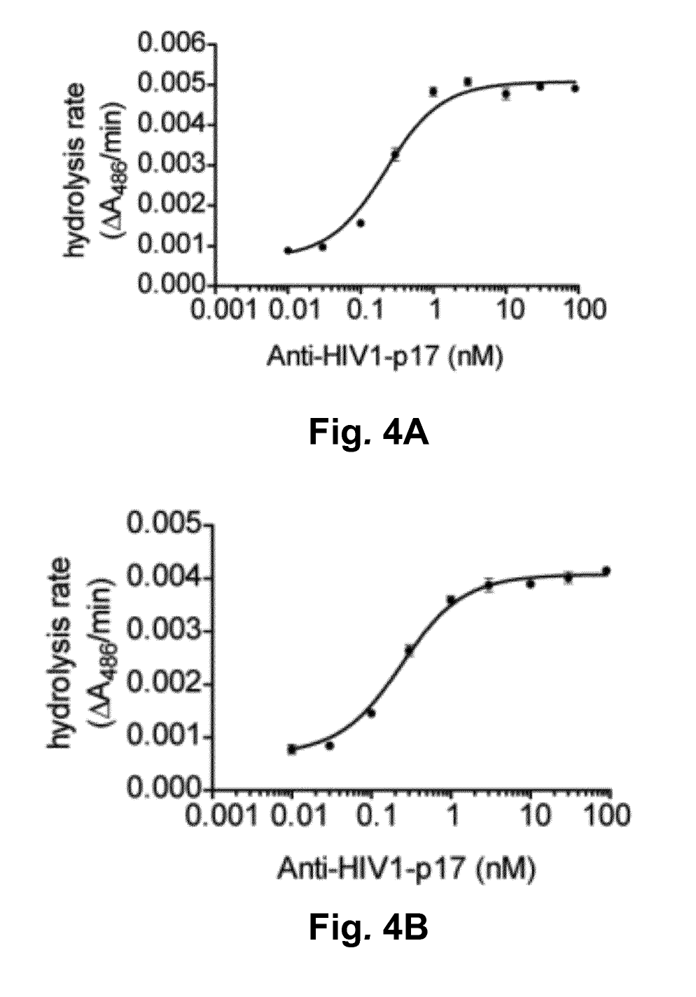

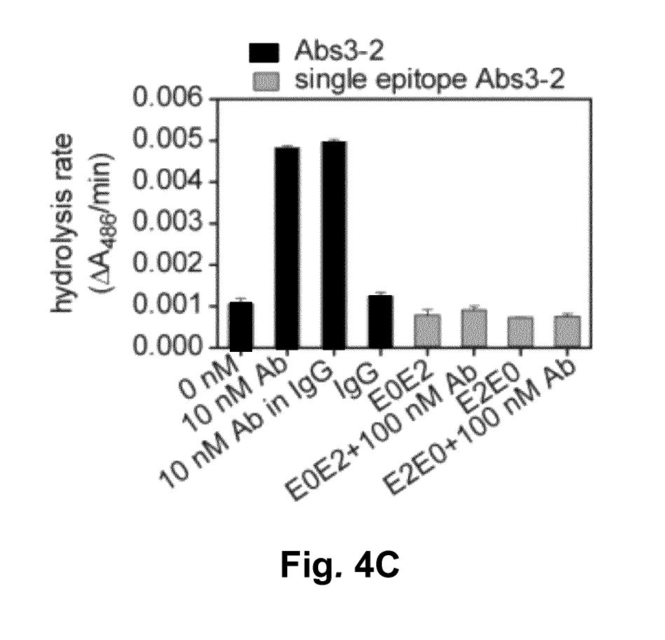

However, combination of the E104D mutation in β-lactamase and single point mutations in BLIP yielded several sensor variants that showed an increase in enzymatic activity upon addition of the target antibody (FIG. 3). The variants that displayed substantial activity in the absence of antibody typically showed only a modest increase in activity upon antibody binding. The two most promising variants, Abs-3-E104D/E31A (Abs3-1; SEQ ID NO: 22) and Abs-3-E104D/F142A (Abs3-2; SEQ ID NO: 37), were further characterized, as these showed low background activity and a 5-6 fold increase in enzyme activity. To determine the affinity of each sensor for their target antibody, the rate of nitrocefin hydrolysis was measured as a function of antibody concentration using 100 μM of sensor (FIGS. 4A-B).

Fitting these curves assuming a 1:1 binding model yielded dissociation constants of 0.17±0.03 nM for Abs3-1 (SEQ ID NO: 22) and 0.19±0.02 nM for Abs3-2 (SEQ ID NO:37), which is 200-fold lower than that of a single epitope peptide (K_{d}=42 nM). The bivalent interaction between sensor and antibody thus not only provides a convenient switching mechanism, but also results in a substantial increase in overall affinity. In further testing of this mechanism we verified that both epitopes are required for antibody-induced activation. Two variants of Abs3-2 were generated in which either the epitope next to the enzyme (Abs-3E0E2; SEQ ID NO: 38) or the epitope adjacent to the BLIP domain were deleted (Abs-3E2E0; (SEQ ID NO: 39). Indeed, both variants showed a low enzymatic activity and none of them showed any increase in enzymatic activity up to 100 nM of anti-HIV1-p17 antibody (FIG. 4C). These results confirmed that the activity increase is due to bivalent binding of the antibody to the two epitope sequences. To test the specificity of the sensors, Abs3-2 (and Abs3-1) was also incubated with a random mix of IgG proteins. No significant increase in enzyme activity was observed up to the highest concentration of IgG tested (2 μM). Moreover, the presence of nonspecific IgG's did also not affect the binding of the target antibody, as a similar increase in enzyme activity was observed upon addition of 10 nM anti-HIV1-p17 in the absence and presence of a large excess of IgG mix (0.1 mg/mL, i.e. 660 nM) (FIG. 4C). The latter is important because in serum specific antibodies need to be detected against a background concentration of non-binding antibodies.

### Targeting a Different Antibody by Exchanging Epitopes

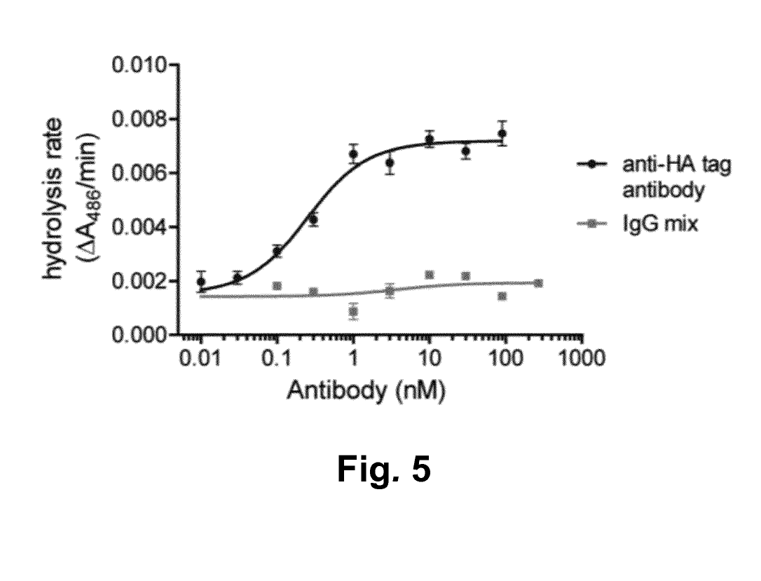

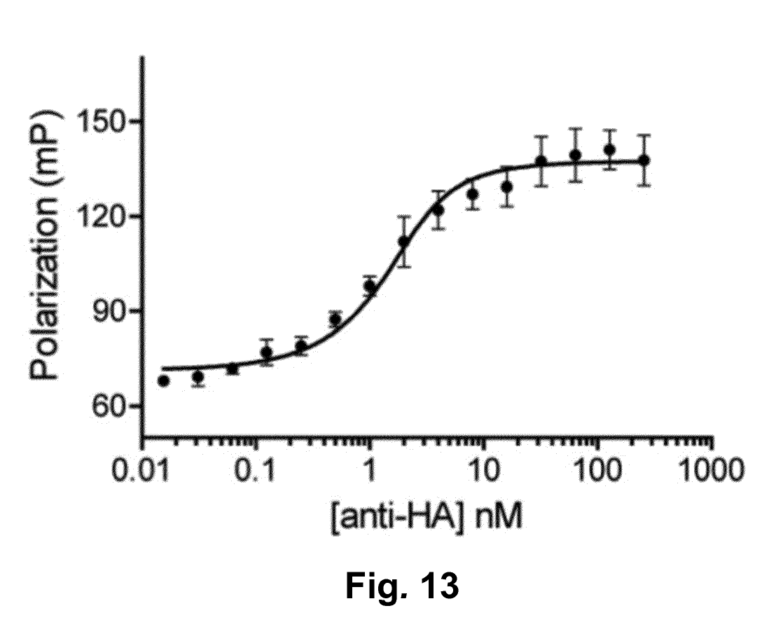

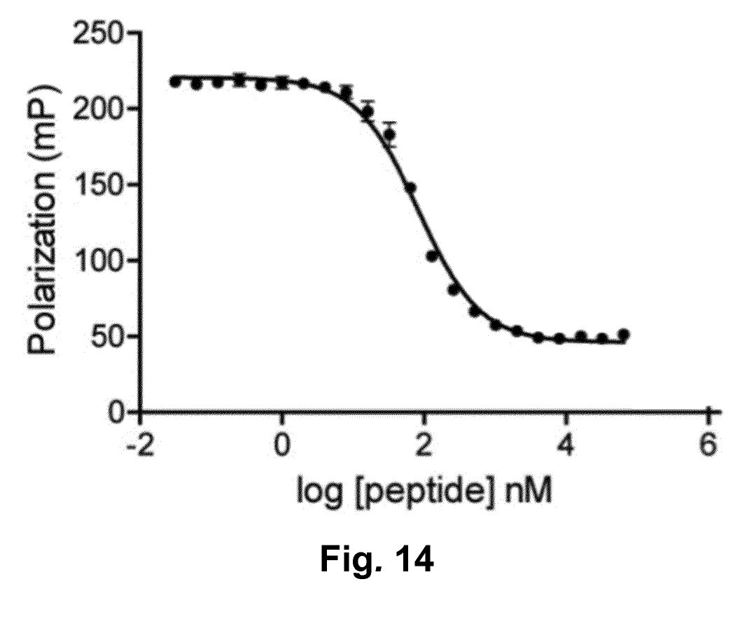

To challenge the modularity of our sensor design we tested whether the epitope sequences could be exchanged for epitope sequences targeting a different antibody. A reporter enzyme targeting an HA-tag-specific antibody (Abs-4; SEQ ID NO: 40) was constructed by replacing the epitope sequences present in Abs3-1 by YPYDVPDYA (SEQ ID NO: 9). The monovalent affinity of the anti-HA-antibody for this peptide was found to be ˜5 nM based on fluorescence polarization titration experiments (FIGS. 13-14), which is similar to that of the anti-HIV1 p17 antibody for the long epitope sequences present in Abs-3 (SEQ ID NO: 21). Although no sensor optimization was performed, Abs-4 (SEQ ID NO: 40) showed very similar sensor properties compared to its parent sensor Abs3-1 (SEQ ID NO:22). Titration of anti-HA antibody again resulted in 7-fold increase in activity and K_{d} of 0.20 nM for the sensor-antibody interaction (FIG. 5). These results show that the framework developed for the exemplary anti-HIV1-p17 antibody can be used to develop β-lactamase reporter enzymes for other antibodies without the cumbersome optimization/screening procedures required by previous protein engineering strategies.

Assays with Fluorescent Substrate

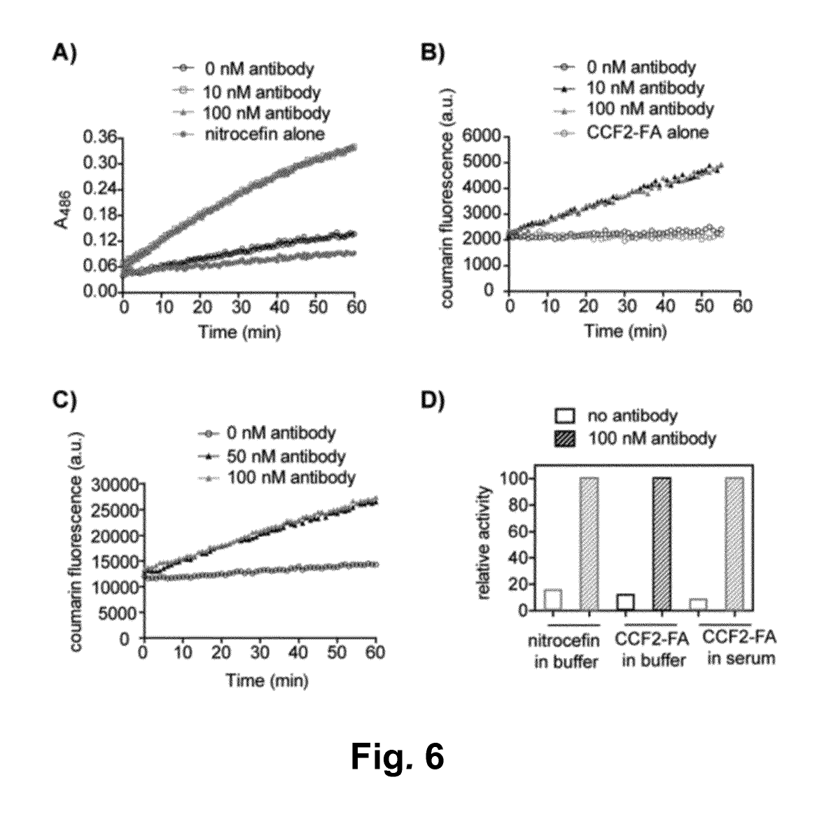

The use of nitrocefin and other colorimetric substrates for our antibody sensors provide a straightforward means to detect sub-nM concentrations of a specific antibody directly by eye. However, assays based on light absorption measurements require relatively high concentrations of substrate. Since the substrate and BLIP compete for the same binding site on β-lactamase, using high substrate concentrations will result in a relatively high background activity. We therefore assessed the performance of Abs3-1 (SEQ ID NO: 22) using the commercially available fluorescent substrate CCF2-FA, which could be used at a 50-fold lower concentration. When this FRET probe is hydrolyzed by the enzyme, a fluorescein molecule (acceptor) is expelled from the probe which results in increase in coumarin fluorescence (donor). Unlike nitrocefin, which even in the absence of sensor is slowly hydrolyzed, CCF2-FA was found to be completely stable providing a low background (FIG. 6B). Moreover, the enzymatic activity of the sensor protein in the absence of target antibody was found to be significantly lower using 1 μM of CCF2-FA compared to assays with 50 μM nitrocefin (FIGS. 6A-B and 6D). Therefore using CCF2-FA as a substrate resulted in an increased dynamic range (9-fold) by suppressing the background reaction. The fluorescent substrate also proved essential to employ the reporter enzyme in serum. Unlike nitrocefin, which was rapidly hydrolyzed even in the absence of any reporter enzyme, CCF2-FA was found to be completely stable in both bovine and human serum. Assays using β-lactamase-E104D (SEQ ID NO: 45) showed that the enzyme is substantially less active in serum compared to PBS, suggesting the presence of inhibitory compounds in serum. To compensate for this decreased activity, assays in serum were done using 5 nM of reporter enzyme. FIG. 6C shows that the dynamic range of the reporter enzyme in serum is at least as high as observed in buffer, showing a 10-fold increase in enzyme activity upon addition of 50 nM of target antibody.

### Thermodynamic Model

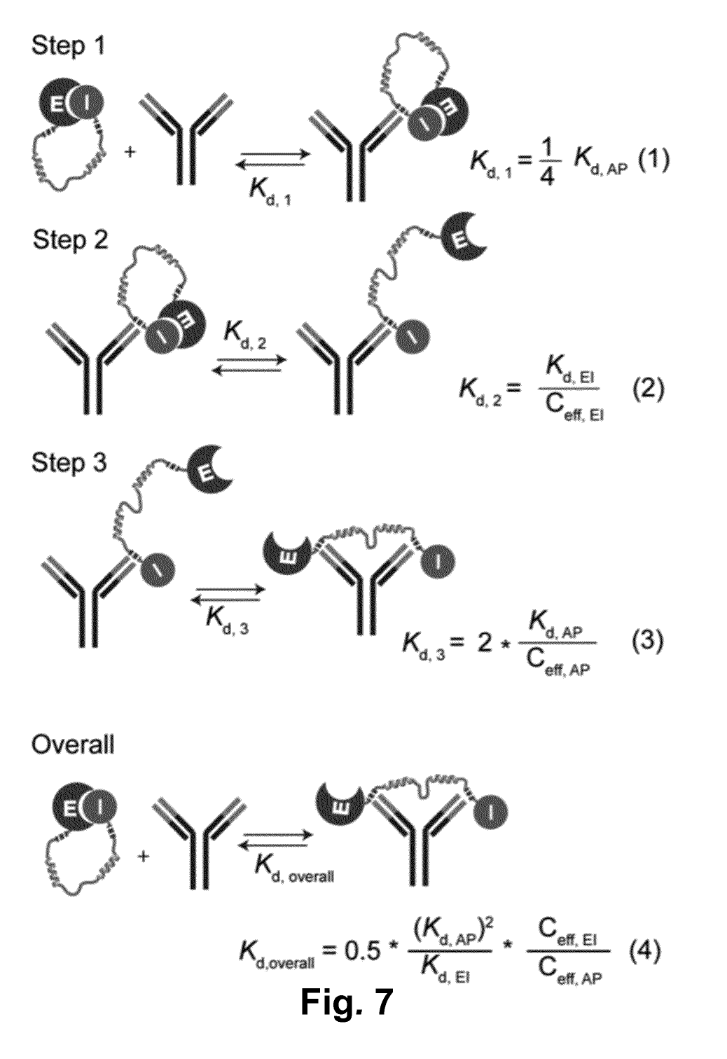

To get a better insight into the factors that determine the performance of these sensors, a thermodynamic model was derived that describes the bivalent binding between the antibody and the sensor in 3 steps (FIG. 7). The first reaction is binding of one of the antigen binding domains to one of the epitope sequences. This equilibrium is determined by the affinity between the epitope and the antigen binding domain (K_{d, AP}). The second step is dissociation of the β-lactamase-inhibitor complex. This equilibrium depends on the affinity of the enzyme-inhibitor complex (K_{d, EI}) and the effective concentration of BLIP relative to its enzyme domain (C_{eff, EI}), which in turn will depend on the linker length and stiffness and the distance that the linker bridges in the complex form. This equilibrium determines how much of the enzyme is inhibited in absence of antibody. If K_{d, EI}>C_{eff,EI} than most of the sensor will be already active in the absence of antibody, and a poor sensor is obtained. This was the case for Abs1 and some of the mutants depicted in FIG. 3. (e.g. the variant with W150A in the BLIP domain). To have a useful sensor K_{d, EI}/C_{eff, E} Should be preferably be below 0.2, which corresponds to 83% inhibition in the absence of the antibody. The final reaction step is formation of the second epitope-antibody interaction, which again is a function of an effective concentration term (C_{eff, AP}) and the affinity of the epitope-antibody interaction (K_{d, AP}). The overall dissociation constant of the reporter enzyme for its target antibody is the product of the equilibrium constants for the three steps and is described by Eq. 4 in FIG. 7.

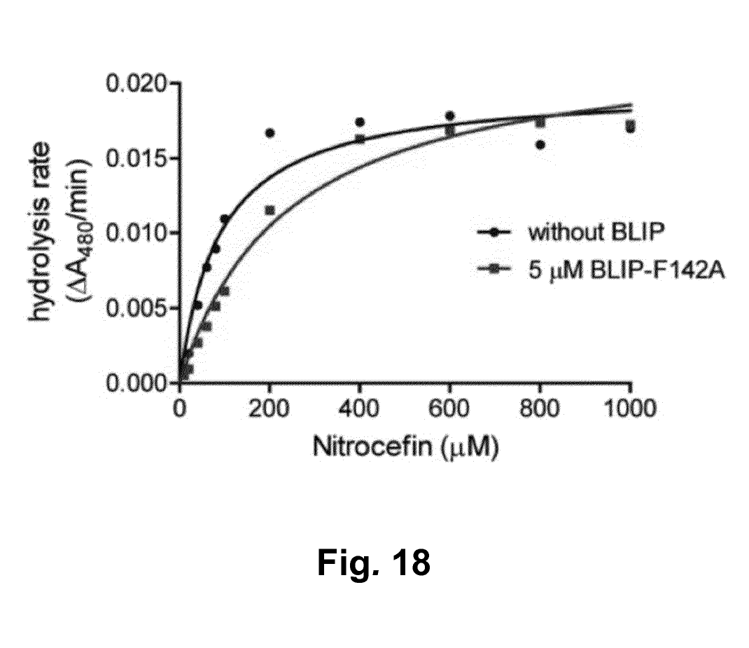

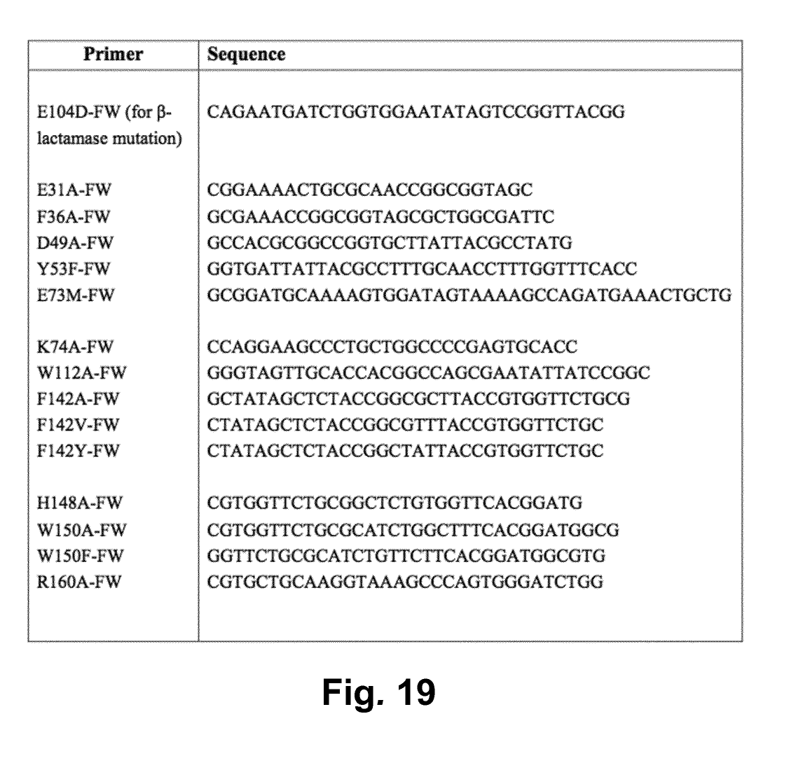

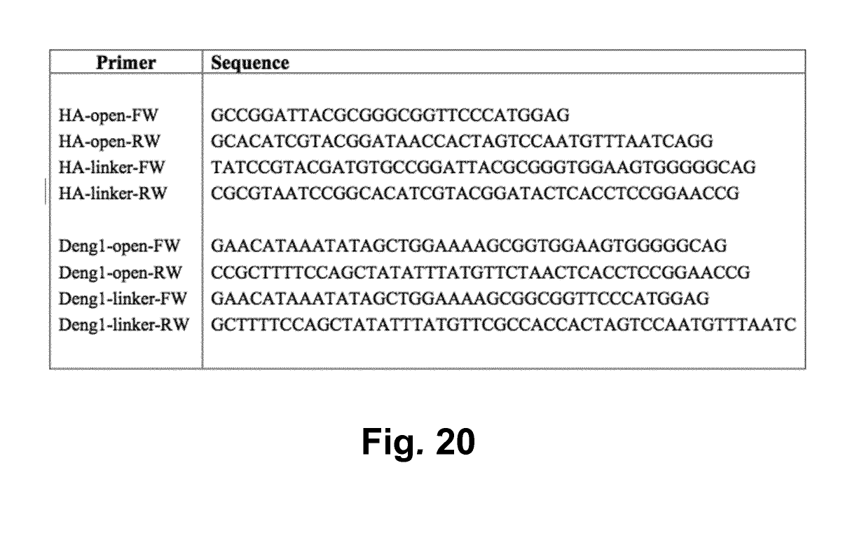

Of the four parameters that determine K_{d-overall}, two can be determined independently. Fluorescence polarization was used to determine the affinity between a fluorescently labeled epitope peptides and the anti-HIV1 antibody, yielding a K_{d, AP} of 42 nM. The dissociation constant for the enzyme-inhibitor pairs was obtained by from enzyme kinetics experiments by determining the competitive inhibition constant for BLIP-E31A and BLIP-F142A and β-lactamase E104D, yielding Kᵢ values of 2.11 μM for BLIP-E31A is and 2.94 μM for BLIP-F142A (FIG. 18). Using K_{d, AP}=42 nM and K_{d, EI}=2.9 μM, and K_{d, overall}=0.17 nM, allows one to calculate C_{eff, EI}/C_{eff, AP}) to be 0.56. Since this value is close to 1, this means that the linker itself does not preferentially stabilize either the open or the closed complex, but that the equilibrium between open and closed forms depends on the relative affinities of the enzyme-inhibitor interaction and the epitope-antibody interaction.

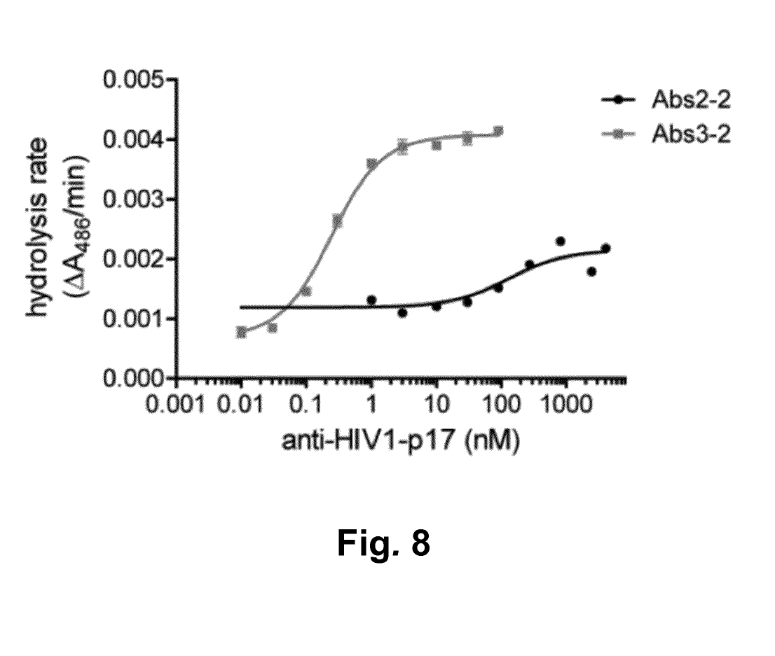

Eq. 4 (FIG. 7) predicts that the overall affinity of the sensor strongly depends on strength of the antibody-epitope interaction. To test this, we changed the large epitope used in Abs3-2 (SEQ ID NO: 37) for the shorter WEKIRLR epitope, which has a K_{d} of 3.3 μM. The enzyme activity of this variant (Abs2-2; SEQ ID NO: 20) also increased upon addition of antibody, but the increase was only 1.8 fold (FIG. 8). Moreover, the dissociation constant of the reporter enzyme was increased to 108 nM. This 635-fold increase is roughly consistent with Eq. 4 (FIG. 7), which would predict in increase of (3.3 E⁻⁶/4.2 E⁻⁸)²=1900-fold for this substitution. The model also explains the very modest increase in enzyme activity. The activity of Abs2-2 (SEQ ID NO: 20) in the absence of its target antibody is described by Eq. 5-1 (equal to Eq. 2 in FIG. 7).

K_{closed-open,1}=K_{d(EI)}/C_{eff,(EI)} (5-1)

Since this equilibrium depends only on K_{d(EI)} and C_{eff(EI)}, the background activity of Abs2-2 (SEQ ID NO: 20) and Abs3-2 (SEQ ID NO: 37) are very similar. What is different is the equilibrium between the closes and open states of the reporter enzyme in the presence of the saturating amounts of the target antibody. This equilibrium is determined by steps 2 and 3 (FIG. 7). Eq. 5-2 describes the equilibrium constant for the product of these two steps 2 and 3.

K_{closed-open,2}=0.5*K_{d,(EI)}/K_{d(AP)}*C_{eff,(AP)}/C_{eff(EI)} (5-2)

Using C_{eff(EI)}/C_{eff}(AP)=0.56, K_{d(EI)}=2.9 μM, and K_{d}(AP)=3.3 μM yields K_{closed-open}=0.78. So even in the presence of saturating concentrations of antibody a significant amount (60%) of the Abs 2.2 (SEQ ID NO: 20) reporter enzymes is still in the closed state, which explains the modest increase in enzyme activity observed for this reporter enzyme. The reasonable agreement between the predictions of our thermodynamic model and the experimental results suggest that in first approximation the sensor properties of the β-lactamase-BLIP system can be predicted based on the relative stabilities of the epitope-antibody and the β-lactamase-inhibitor complexes. This model provides useful guidelines for the construction of new reporter enzymes, either using the present β-lactamase-BLIP system or systems based on alternative enzyme-inhibitor pairs. E.g. ideally, K_{closed-open,1} (in the absence of the antibody) should be below 0.2, whereas K_{closed-open, 2} (in the presence of antibody) should be above 5.

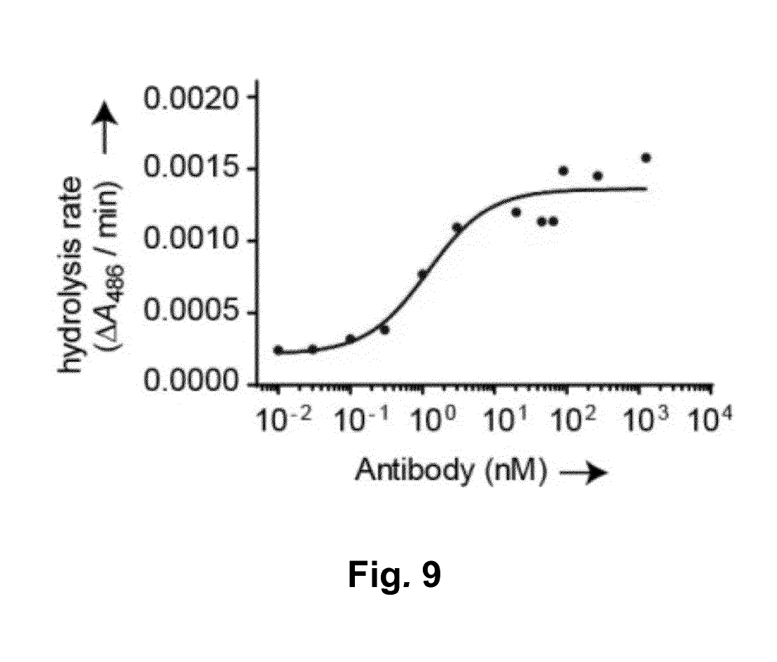

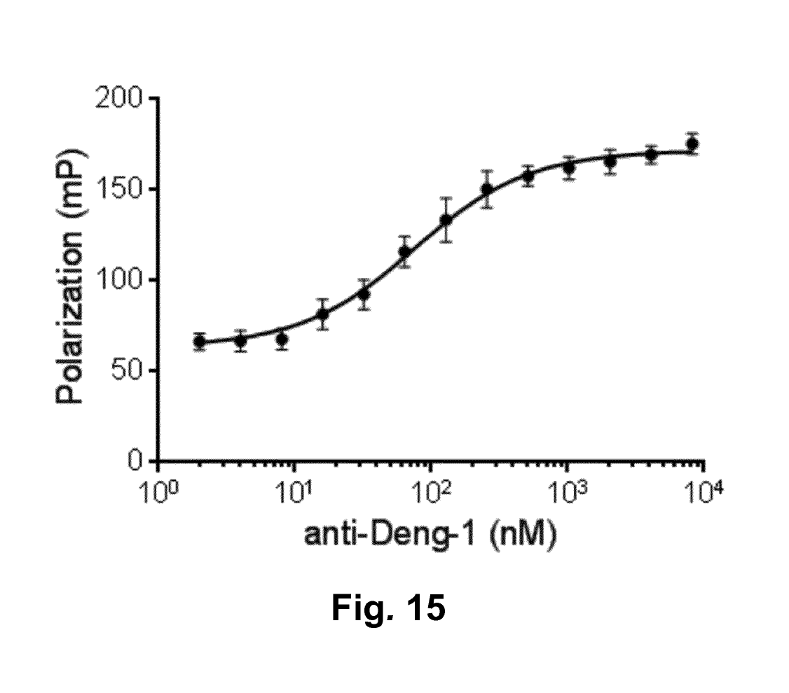

To further challenge the modularity of the biosensor design we tested whether the original epitope sequences could be exchanged for epitope sequences targeting a Dengue type I specific antibody. This reporter enzyme Abs-5 (SEQ ID NO: 42) was constructed by replacing the epitope sequences present in Abs-3-1 (SEQ ID NO: 36) by EHKYSWKS (Abs-5). Fluorescence polarization titration experiments yielded a K_{d} values of 70 nM for the monovalent peptide-antibody interaction (K_{d, AP}) (FIG. 15). Although no sensor optimization was performed, Abs-5 (SEQ ID NO: 42) showed very similar sensor properties compared to its parent sensor Abs-3-1 (SEQ ID NO: 36). A 7-fold increase in enzymatic activity was observed upon titration of Dengue type I antibody to Abs-5 (SEQ ID NO:42), consistent with a K_{d} of 1.13 nM±0.46 (FIG. 9). Although the antibody affinity is slightly attenuated in Abs-5, this K_{d} still reflects a 60-fold increase in affinity compared to the monovalent interaction between antibody and peptide epitope. These results show that the framework developed for the HIV1-p17 antibody allows the antibody specificity to be changed merely by replacing the epitope sequences without the subsequent sensor optimization required by other by protein engineering strategies.

### EXPERIMENTAL SECTION

### Cloning and Mutagenesis

Synthetic DNA sequences encoding the Abs-1 (SEQ ID NO:12), BLIP and linker 2 (linker with longer epitope sequence, E2) were ordered from Genscript (Piscataway, USA). Inhibitor peptide in the Abs-1 was replaced with BLIP sequence by cloning with NcoI and EcoRI restriction enzymes to generate Abs-2 (SEQ ID NO: 13). Linker 1 (linker with short epitope sequence, E1) in the Abs-2 construct was replaced with the linker 2 sequence by cloning with SpeI and NcoI restriction enzymes to generate Abs-3 (SEQ ID NO: 21) sensors. Abs-4 (SEQ ID NO: 40) and Abs-5 (SEQ ID NO: 42) were constructed from Abs-3 using a strategy described previously (Quan, J. and Tian, J. (2009) Circular polymerase extension cloning of complex gene libraries and pathways. PLoS One 4, e6441). Briefly, the Abs-3 (SEQ ID NO: 21) vector was opened with PCR using primers HA-open-FW and HA-open-RW for Abs-4 and Dengl-open-FW and Dengl-open-RW for Abs-5. The linker part of Abs-3 without HIV1 epitopes was PCR-amplified with the HA-linker-FW and HA-linker-RW primers for Abs-4 and Dengl-linker-FW and Dengl-linker-RW for Abs-5. This PCR-generated linker contains sequences encoding for either the HA-epitope or Dengue-1 epitopes and sequences that overlap with the opened vector. After agarose gel purification, both opened vector and linker were mixed and PCR was performed. The PCR mixture was treated with DpnI to remove any remaining parental DNA. Transformation and then sequencing of colonies showed successful exchange of the epitope sequences. All constructs were cloned into pET29a vectors. The QuikChange site-directed mutagenesis kit (Stratagene) was used in accordance with the manufacturer's instructions to introduce the mutations of interest. All cloning and mutagenesis results were confirmed by DNA sequencing (BaseClear, Leiden, The Netherlands).

### Protein Expression and Purification

All proteins were expressed and purified using standard protocols. Briefly, E. coli BL21(DE3) cells were transformed with the appropriate pET29a vector. The bacteria containing plasmid DNA was grown in LB (2 L) media at 37 degrees Celsius and induced at OD₆₀₀˜0.6 with isopropyl-β-D-thiogalactoside (IPTG; 0.3 mM). Induced cells were grown overnight at 15 degrees Celsius, and harvested for 10 min at 8000 g. The protein is located in periplasm that was extracted by osmotic shock method. The bacterial pellet was resuspended in 100 mL 30 mM Tris/HCl (pH 8.0), 20% (w/v) sucrose, and 1 mM EDTA, and incubated at room temperature for 10 min under continuous shaking. After centrifugation for 10 min at 8000 g, the pellet was resuspended in 100 mL of ice-cold 5 mM MgSO₄. After incubation for 10 min at 4 degrees Celsius with continuous shaking, the suspension was centrifuged for 20 min at 12000 g. The supernatant (contains the periplasmic protein fraction) was adjusted to pH 7.4 by adding 2 mL of 1 M Tris/HCl (pH 7.4). The supernatant was first loaded onto an immobilized metal-affinity column packed with His-bind resin in accordance with the manufacturer's instructions (Novagen). The eluted fractions were further purified on a Strep-Tactin superflow column (IBA) according to the instructions of the supplier. The purified proteins were dialyzed against 50 mM Tris/HCl (pH 7.1) containing 150 mM NaCl using 3.5 kD MWCO membranes (Spectra/Por 3). The proteins were quantified using a Nanodrop ND-1000 spectrophotometer (Wilmington, USA) by using extinction coefficient at 280 nm (calculated from protein sequence using http://web.expasy.org/protparam/). Protein aliquots were stored at −80 degrees Celsius.

### Activity Assays

Antibodies were purchased from commercial sources, anti-HIV-1-p17 (clone 32/1.24.89) from Zeptometrix, HA monoclonal antibody (clone 2-2.2.14) from Thermo Scientific and anti-Dengue virus type I antibody (clone 15F3-1) from Merck Millipore. Nonspecific IgG mix isolated from human serum was purchased from Sigma-Aldrich. Fetal bovine serum (FBS) was purchased from BioChrom AG, Germany. For activity assays, antibody was incubated with sensor proteins for 15 min at room temperature, and then 50 μM of nitrocefin was added. For responsive sensors, 100 μM of sensor was treated up to 100 nM of antibody. For non-responsive sensors 300 μM of sensor was treated with up to 200 nM antibody and incubated for 1 h. The assays were performed in 50 mM phosphate buffer (pH 7.0, containing 100 mM NaCl and 1 mg/mL BSA). Measurements were performed in 96-well plates. Both absorbance (486 nm) with nitrocefin substrate, and fluorescence with CCF2-FA (Ex. 409 nm and Em. 447 nm) was recorded on Safire2 spectrofluoremeter (Tecan). The data were plotted and analyzed using Graphad Prism5 software. Assays using β-lactamase-E104D showed that the enzyme is substantially less active in serum compared to PBS, suggesting the presence of inhibitory compounds in serum. To compensate for this decreased activity, assays in serum were done using 5 nM of reporter enzyme.

### Antibody Titrations and K_{d} Measurements

Antibody titrations were performed as mentioned above, initial 10 min data was used for calculating hydrolysis rate of the substrate. The data, activity change with the antibody concentration, was fit to Eq. 6 to obtain dissociation constants. In Eq. 6, A and B are constants, and [sensor] and [Ab] are the total sensor and antibody concentrations, respectively.

Hydrolysis rate=A×(([sensor]+[Ab]+K_{d})−(([sensor]+[Ab]+K_{d})²−4[Ab][sensor])^{1/2})+B (6)

### Enzyme Inhibition Assays and Ki Determination:

To a 100 μM of β-lactamase-E104D mutant 5 μM BLIP protein was added and incubated for 2 h at 30 degrees Celsius. Aliquots of enzyme alone or enzyme-inhibitor complex was pipetted into a 96-well plate. To these samples different concentration of nitrocefin (10 to 1000 μM) was added. Absorbance at 486 nm was recorded over the time. Initial 10 min data was used for determining the hydrolysis rate which then fit into Michaelis-Menten equation to obtain K_{M} of the β-lactamase-E104D with or without BLIP. Inhibition constant, Kᵢ, of the BLIP was obtained using Eq. 7. In Eq. 7 K_{m (+BLIP)} and K_{M} are the Michaelis-Menten in the presence and absence of BLIP, respectively.

K_{m(+BLIP)}=Kₘ(1+([BLIP]/Kᵢ)) (7)

### Supplemental Information

### Peptide Synthesis

The peptides were synthesized from C- to N-terminus on 200 μmol scale using manual solid phase peptide synthesis. Rink Amide MBHA resin (340 mg, loading: 0.59 mmol/g) was put in a 20 mL syringe with filter, and allowed to swell in NMP for 30 minutes on a shaker. A 400 mM stock solution of HCTU was prepared. A 20% piperidine in NMP solution was used for Fmoc deprotection and a 3/1/1 NMP/Ac₂O/pyridine solution was used for capping. All amino acids were dissolved in NMP, creating stock solutions of 200 mM. DIPEA could be used directly out of the bottle. The Fmoc-group was removed using 20% piperidine (2 times 5 minutes on a shaker) followed by an NMP wash (3 times, shake syringe by hand for approximately 30 seconds). Before coupling, amino acids were preactivated (5 minutes) using 4 mL of 200 mM amino acid solution (4 eq.) with 2 ml of 400 mM HCTU (4 eq.). 279 μL of DIPEA (8 eq.) was added and the amino acid was coupled for 30 minutes on a shaker, followed by a NMP wash. Capping was performed using 3/1/1 NMP/Ac₂O/Pyridine solution (2 times 5 minutes on a shaker) followed by an NMP wash. The first amino acid (on the C-terminus) was coupled 2 times for 45 minutes. Because the length of the peptide, synthesis was sometimes stopped halfway (just before the capping step), washed 2 times with DCM and dried under vacuum for 30 minutes. The unfinished peptide was stored in the fridge overnight. When synthesis was continued the resin was again swollen in NMP for 30 minutes, followed by a capping step. When the peptide was fully synthesized, an extra deprotection and capping step were performed to protect the N-terminus, yielding an acetylated N-terminus. Then again a NMP wash (3 times) and a DCM wash (2 times) were performed and the peptide (on the resin) was dried under vacuum. Cleavage was accomplished using 5 mL 95/2.5/2.5 TFA/H₂O/TIS and shaking for 3 hours on a shaker. The cleaved peptides were transferred to a 50 mL Falcon tube. The cleaved and deprotected peptide was precipitated by adding diethylether (up to 50 mL), shaken on a vortex and stored in a −30 degrees Celsius freezer for at least 2 hours. The ethereal layer was decanted off after centrifugation (2,000 rpm for 10 min), this step was repeated and the remaining diethylether was evaporated by storing the open Falcon tube in a fume hood for 30 minutes. Peptides were dissolved in H₂O (+0.1% TFA) and ACN, starting with a ratio of 95/5 and adding more ACN if not well dissolved. The solutions were filtered through a 0.45 μm filter using PALL Acrodisc syringe filters with supor membrane. For characterization, LC-MS was performed on the filtrate using a LC-MS (Shimadzu SCL-10 AD VP series HPLC coupled to a diode array detector (Finnigan Surveyor PDA Plus detector, Thermo Electron Corporation) and an Ion-Trap S3 (LCQ Fleet, Thermo Scientific)). A gradient of 2-70% of ACN in H₂O (+0.1% TFA) was used. Purification was performed on prep-RP-HPLC (Shimadzu) using a C18-column using 10 to 50% ACN in H₂O (+0.1% TFA) with a flow rate of 15 mL/min. All peptides were acetylated at N-terminus and amidated at the C-terminus.

### Coupling of Fluorescein to Thiol Group of the Peptide:

10 mg of the thiol-containing peptide (1 eq.) and TCEP (2 eq.) were dissolved in pH 7.0 HBS buffer (100 mM HEPES+NaCl 100 mM). The pH was adjusted to 7.0 and then fluorescein-5-maleimide (Invitrogen, Cat#F-150) (1.5 eq.) was added and stirred at RT for overnight. The product was purified by prep-RP-HPLC. Analysis and characterization was done with LC-MS. The ESI-MS indicated that part of the product had been converted to a +18 Da derivative, which results from hydrolytic ring opening of a maleimide-derived succinimide group. Since this modification is unlikely to affect the binding properties of the peptides, we did not attempt to separate both species.

### HIV1-p17 Short Epitope

### WEKIRLR-GGG-C (SEQ ID NO:10)

LC-MS: m/z [M+2H]2+ Calcd. 658.78 Da Obsd. 658.75 Da. [M]+ Calcd. 1315.55 Da Obsd. 1315.92 Da.

### WEKIRLR-GGG-C(Fluorescein)

LC-MS: m/z [M+2H]2+ Calcd. 872.97 Da Obsd. 872.58 Da. [M]+ Calcd. 1743.92 Da Obsd. 1743.92 Da.

And [M+H2O+H]2+ Calcd. 881.47 Da Obsd. 881.33 Da. [M+H2O]+ Calcd. 1761.93 Da Obsd. 1761.92 Da

HIV1-p17 longer epitope

### ELDRWEKIRLRP (SEQ ID NO:3)

LC-MS: m/z [M+3H]3+ Calcd. 551.64 Da Obsd. 551.75 Da. [M+2H]2+ Calcd. 826.96 Da Obsd. 826.83 Da. [M+H]+ Calcd. 1652.92 Da Obsd. 1652.92 Da.

### ELDRWEKIRLRP-GGG-C (SEQ ID NO:2)

LC-MS: m/z [M+3H]3+ Calcd. 643.08 Da Obsd. 643.25 Da. [M+H]2+ Calcd. 963.61 Da Obsd. 963.83 Da. [M+H]+ Calcd. 1927.21 Da Obsd. 1927.08 Da.

### ELDRWEKIRLRP-GGG-C(Fluorescein)

LC-MS: m/z [M+H]2+ Calcd. 1177.95 Da Obsd. 1177.67 Da. [M+2H]3+ Calcd. 785.53 Da Obsd. 785.58 Da. [M+3H]4+ Calcd. 589.40 Da Obsd. 589.58 Da.

### HA-Tag Peptide

### YPYDVPDYA (SEQ ID NO:5)

LC-MS: m/z [M+2H]2+ Calcd. 572.61 Da Obsd. 572.50 Da. [M]+ Calcd. 1143.20 Da Obsd. 1143.75 Da.

### YPYDVPDYA-GGG-C (SEQ ID NO:4)

LC-MS: m/z [M+2H]2+ Calcd. 709.76 Da Obsd. 709.58 Da. [M]+ Calcd. 1417.50 Da Obsd. 1417.75 Da.

### YPYDVPDYA-GGG-C(Fluorescein)

LC-MS: m/z [M+3H]3+ Calcd. 616.30 Da Obsd. 616.00 Da. [M+2H]2+ Calcd. 923.44 Da Obsd. 923.58 Da. [M+H]+ Calcd. 1846.88 Da Obsd. 1846.67 Da.

And [M+H2O+2H]3+ Calcd. 621.97 Da Obsd. 621.92 Da. [M+H2O+H]2+ Calcd. 932.45 Da Obsd. 932.58 Da.

### DEN1 Peptide

### EHKYSWKS-GGG-C (SEQ ID NO:6)

LC-MS: m/z [M+3H]3+ Calcd. 460.54 Da Obsd. 461.00 Da. [M+2H]2+ Calcd. 690.31 Da Obsd. 690.75 Da. [M+H]+ Calcd. 1379.62 Da Obsd. 1379.83 Da.

### EHKYSWKS-GGG-C(Fluorescein)

LC-MS: m/z [M+4H]4+ Calcd. 452.43 Da Obsd. 452.92 Da. [M+3H]3+ Calcd. 602.90 Da Obsd. 603.33 Da. [M+2H]2+ Calcd. 903.85 Da Obsd. 904.17 Da.

and [M+H2O+4H]4+ Calcd. 456.93 Da Obsd. 457.42 Da. [M+H2O+3H]3+ Calcd. 608.90 Da Obsd. 609.33 Da. [M+H2O+2H]2+ Calcd. 912.85 Da Obsd. 913.50 Da.

### Peptide Binding Assays Using Fluorescence Polarization

To determine the affinity of the antibodies for their peptide epitopes, a GGGC sequence was introduced at the C-termini of the epitope sequences. The C-terminal cysteine was used to attach a fluorescein by reacting the cysteine with maleimide-functionalized fluorescein. Binding of antibody to the fluorescently-labeled peptide results in an increase in fluorescence polarization. Eq. 8 was used to fit the polarization as a function of the concentration of antigen binding domains, yielding the dissociation constant for the interaction. In this analysis it was assumed that binding of peptide to each of the antigen binding domains was independent.

$$A = {A}_{f} + ( {A}_{b} - {A}_{f} ) * \frac{( [ P ] + {K}_{d} + [ Ab ] ) - \sqrt{{( [ P ] + {K}_{d} + [ Ab ] )}^{2} - 4 [ P ] [ Ab ]}}{2 [ P ]} \qquad ( 8 )$$

In Eq. 8, A is the measured polarization, A is the polarization of the free peptide, A_{b} is polarization value of the bound peptide, [P] is the peptide concentration and [Ab] is the concentration of antigen binding domains.

FIG. 10 shows titration of 10 nM (WEKIRLR-GGG-C(fluorescein)) with anti-HIV1-p17 antibody monitored using fluorescence polarization. The data were fit to Eq. 8 yielding a K_{d} of 3.3±0.2 μM.

### Affinity of the Long HIV1-p17 Epitope (ELDRWEKIRLRP)

First we determined the affinity of the anti HIV1-p17 antibody by titration of the antibody to 10 nM of fluorescein-labeled peptide (ELDRWEKIRLRP-GGG-C(fluorescein)). This titration yielded a K_{d} of 24±3 nM (FIG. 11). To test whether the fluorescein label influences the interaction with the antibody, we also performed a competition assay in which a fixed concentration of antibody and fluorescently-labeled peptide was titrated with non-fluorescent peptide (ELDRWEKIRLRP; SEQ ID NO:3). The competitive titration was fit to Eq. 9 to yield an EC50 (FIG. 12). This EC50 was subsequently used to calculate the affinity of the antibody for the non-labeled peptide using Eq. 10, yielding a Kd1 value of 42±1 nM.

$$\begin{matrix}A = {A}_{i} + \frac{( {A}_{i} - {A}_{f} )}{( 1 + {10}^{( log [ P ] - logEC 50 )} )} & ( 9 ) \\ log EC 50 = log ( {10}^{{Kd}_{1}} * ( \frac{1 + [ {P}_{fl} ]}{{Kd}_{2}} ) ) & ( 10 )\end{matrix}$$

with Kd₁ is the dissociation constant for the non-labeled peptide, Kd₂ the dissociation constant for the fluorescently labeled peptide, [P] the concentration of the unlabeled peptide, and [P_{fl}] the concentration of the fluorescent peptide.

### Affinity of HA-Tag Epitope Antibody Interaction

First we determined the affinity of the HA-tag antibody by titration of the antibody to 2 nM of fluorescein-labeled peptide (YPYDVPDYA-GGG-C(fluorescein)). This titration showed tight binding and yielded a K_{d}=0.58±0.22 nM (FIG. 13). To test whether the fluorescein label influences the interaction with the antibody, we also performed a competition assay in which a fixed concentration of antibody and fluorescently-labeled peptide was titrated with non-fluorescent peptide (YPYDVPDYA; SEQ ID NO:5). The competitive titration was fit to Eq. 9 to yield an EC50 (FIG. 14). This EC50 was subsequently used to calculate the affinity of the antibody for the non-labeled peptide using Eq. 10, yielding a K_{d1} value of 4.5±1 nM.

### Affinity of Epitope-Antibody Interaction of Dengue Type I Specific Antibody

FIG. 15 shows titration of 10 nM (EHKYSWKS-GGG-C(fluorescein)) with anti-Dengue-1 antibody monitored using fluorescence polarization. The data were fit to Eq. 8 yielding a K_{d} of 70±13 nM.

Characterization of Abs-3 Variants with Single Point Mutations in Lactamase or BLIP

FIG. 16 shows enzymatic activity of Abs-3 mutants (0.3 nM) in the presence and absence of 200 nM of the anti-HIV1-p17 antibody. Antibody and sensor were incubated for 1 h at RT. Then 50 μM of nitrocefin was added and the measurement was started immediately. The assay was performed in pH 7.0 phosphate buffer (50 mM) that contains NaCl (100 mM) and BSA (1 mg/mL).

### Determination of Inhibition Constants of BLIP Mutants-Lactamase-E104D

5 μM BLIP protein was added to 100 μM of beta-lactamase-E104D and incubated for 2 h at 30 degrees Celsius. Aliquots of enzyme alone or enzymeinhibitor complex were pipetted into a 96-well plate. To these samples different concentrations of nitrocefin (10 to 1000 μM) were added. The hydrolysis rate was determined by monitoring the increase in absorbance at 486 nm for 10 minutes. Non-linear least square fitting of the hydrolysis rates as a function of nitrocefin concentration using the Michaelis-Menten equation was used to determine K_{M} values in the absence and presence of BLIP. The Ki was calculated using Eq. 7, which represent the relation between the 2 K_{M} values, the inhibitor concentration and K; for a competitive inhibitor.

Other examples, results and/or embodiments can be found in the U.S. Provisional Patent Application 61/706,186 filed Sep. 27, 2012 to which this application claims priority and which is hereby incorporated to this application in its entirety.

What is claimed is:

1. An antibody detection method, comprising:
using a biosensor for detecting an antibody,
wherein said biosensor comprises an enzyme covalently linked to an inhibitor protein via a peptide linker having two epitopes at the ends of said peptide linker, and
wherein said biosensor is defined by two equilibrium constants, K_{closed-open,1} and K_{closed-open,2} that describe the equilibrium between a closed and an open state of said biosensor in the absence and presence, respectively, of said antibody according to:
K_{closed-open,1}=K_{d(EI)}/C_{eff,(EI)}, and
K_{closed-open,2}=0.5*K_{d,(EI)}/K_{d(AP)}*C_{eff,(AP)}/C_{eff(EI)}
wherein:
K_{d(AP)} is an intermolecular dissociation constant of a monovalent binding of said antibody and said epitope,
K_{d(EI)} is an intermolecular dissociation constant of the binding of said enzyme and said inhibitor protein,
C_{eff(EI)} is an effective concentration of said inhibitor protein in proximity of said enzyme, and
C_{eff(AP)} is an effective concentration of a free epitope in proximity of the remaining antigen-binding domain of said antibody.

2. The method as set forth in claim 1, wherein said K_{closed-open,1} is smaller than said K_{closed-open,2}.

3. The method as set forth in claim 1, wherein said K_{closed-open,1} is less than 3.

4. The method as set forth in claim 1, wherein said K_{closed-open,1} is larger than 0 and less than 0.2.

5. The method as set forth in claim 1, wherein said K_{closed-open,2} is greater than 0.2.

6. The method as set forth in claim 1, wherein said K_{closed-open,2} is larger than 0.2 and less than 10⁶.

7. An in vitro antibody-detecting method, comprising:
(a) contacting a sample with a biosensor; said biosensor comprising:
(i) a reporter enzyme;
(ii) an inhibitor domain having affinity for said reporter enzyme;
(iii) at least two epitopes, each epitope having affinity for said antibody; and
(iv) a linker;
(b) determining the activity of said reporter enzyme in the presence of a sample; and
(c) attributing the activity of said reporter enzyme in the presence of said sample to the quantitative or qualitative presence or absence of an antibody,
wherein, in the absence of said antibody, said biosensor is in a closed, inactive state in which at least some of said reporter enzyme forms an intramolecular complex with said inhibitor domain, and
wherein a bivalent binding between two antigen binding domains present in said antibody and said at least two epitopes present at the ends of said linker between said reporter enzyme and said inhibitor domain in said biosensor changes the equilibrium between the closed (inactive) and an open (active) state of said biosensor such that the amount of said reporter enzyme that forms an intramolecular complex with said inhibitor domain is decreased.

8. The method as set forth in claim 7, wherein said reporter enzyme is beta-lactamase or a fragment thereof.

9. The method as set forth in claim 7, wherein said inhibitor domain is a beta-lactamase inhibitor protein or a fragment thereof.

10. The method as set forth in claim 7, wherein, in the closed state, said at least some of said reporter enzyme forms an intramolecular complex with said inhibitor domain is at least 30% of said reporter enzyme forms an intramolecular complex with said inhibitor domain.

11. The method as set forth in claim 7, wherein, in the closed state, said at least some of said reporter enzyme forms an intramolecular complex with said inhibitor domain is at least 50% of said reporter enzyme forms an intramolecular complex with said inhibitor domain.

12. The method as set forth in claim 7, wherein, in the closed state, said at least some of said reporter enzyme forms an intramolecular complex with said inhibitor domain is at least 80% of said reporter enzyme forms an intramolecular complex with said inhibitor domain.


SEQUENCE LISTING (71 sequences)

SEQ ID NO: 1 - protein, 7 aa, Unknown
  synthetic contruct
WEKIRLR

SEQ ID NO: 2 - protein, 16 aa, Unknown
  synthetic construct
ELDRWEKIRLRPGGGC

SEQ ID NO: 3 - protein, 12 aa, Unknown
  synthetic construct
ELDRWEKIRLRP

SEQ ID NO: 4 - protein, 13 aa, Unknown
  synthetic construct
YPYDVPDYAGGGC

SEQ ID NO: 5 - protein, 9 aa, Unknown
  synthetic contruct
YPYDVPDYA

SEQ ID NO: 6 - protein, 12 aa, Unknown
  synthetic construct
EHKYSWKSGGGC

SEQ ID NO: 7 - protein, 5 aa, Unknown
  synthetic construct
EAAAK

SEQ ID NO: 8 - protein, 6 aa, Unknown
  synthetic construct
RRGHYY

SEQ ID NO: 9 - protein, 9 aa, Unknown
  synthetic construct
YPYDVPDYA

SEQ ID NO: 10 - protein, 11 aa, Unknown
  synthetic construct
WEKIRLRGGGC

SEQ ID NO: 11 - protein, 11 aa, Unknown
  synthetic construct
ELDRWEKIRLR

SEQ ID NO: 12 - protein, 474 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGWEKIRLRGGSGGSGGSGGSGGSGGSGAEAAAKEAAAKEAAAKEAAAKEAAAKEAAAKAGSGGSGGSGGPQGILGQGSGGSGGSGGSGAEAAAKEAAAKEAAAKEAAAKEAAAKEAAAKAGSGGSGGSGGSGGSGGSGGWEKIRLRGGSGGSHGRRGHYYGEFGGWSHPQFEK

SEQ ID NO: 13 - protein, 633 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGWEKIRLRGGSGGSGGSGGSGGSGGSGAEAAAKEAAAKEAAAKEAAAKEAAAKEAAAKAGSGGSGGSGGPQGILGQGSGGSGGSGGSGAEAAAKEAAAKEAAAKEAAAKEAAAKEAAAKAGSGGSGGSGGSGGSGGSGGWEKIRLRGGSGGSHGAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGFYRGSAHLWFTDGVLQGKRQWDLVGEFGGWSHPQFEK

SEQ ID NO: 14 - protein, 633 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGWEKIRLRGGSGGSGGSGGSGGSGGSGAEAAAKEAAAKEAAAKEAAAKEAAAKEAAAKAGSGGSGGSGGPQGILGQGSGGSGGSGGSGAEAAAKEAAAKEAAAKEAAAKEAAAKEAAAKAGSGGSGGSGGSGGSGGSGGWEKIRLRGGSGGSHGAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGFYRGSAHLAFTDGVLQGKRQWDLVGEFGGWSHPQFEK

SEQ ID NO: 15 - protein, 633 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGWEKIRLRGGSGGSGGSGGSGGSGGSGAEAAAKEAAAKEAAAKEAAAKEAAAKEAAAKAGSGGSGGSGGPQGILGQGSGGSGGSGGSGAEAAAKEAAAKEAAAKEAAAKEAAAKEAAAKAGSGGSGGSGGSGGSGGSGGWEKIRLRGGSGGSHGAGVMTGAKFTQIQFGMTRQQVLDIAGAENCETGGSFGDSIHCRGHAAGAYYAYATFGFTSAAADAKVDSKSQEKLLAPSAPTLTLAKFNQVTVGMTRAQVLATVGQGSCTTWSEYYPAYPSTAGVTLSLSCFDVDGYSSTGAYRGSAHLWFTDGVLQGKRQWDLVGEFGGWSHPQFEK

SEQ ID NO: 16 - protein, 633 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGWEKIRLRGGSGGSGGSGGSGGSGGSGAEAAAKEAAAKEAAAKEAAAKEAAAKEAAAKAGSGGSGGSGGPQGILGQGSGGSGGSGGSGAEAAAKEAAAKEAAAKEAAAKEAAAKEAAAKAGSGGSGGSGGSGGSGGSGGWEKIRLRGGSGGSHGAGVMTGAKFTQIQFGMTRQQVLDIAGAENCETGGSFGDSIHCRGHAAGAYYAYATFGFTSAAADAKVDSKSQEKLLAPSAPTLTLAKFNQVTVGMTRAQVLATVGQGSCTTWSEYYPAYPSTAGVTLSLSCFDVDGYSSTGFYRGSAALWFTDGVLQGKRQWDLVGEFGGWSHPQFEK

SEQ ID NO: 17 - protein, 633 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGWEKIRLRGGSGGSGGSGGSGGSGGSGAEAAAKEAAAKEAAAKEAAAKEAAAKEAAAKAGSGGSGGSGGPQGILGQGSGGSGGSGGSGAEAAAKEAAAKEAAAKEAAAKEAAAKEAAAKAGSGGSGGSGGSGGSGGSGGWEKIRLRGGSGGSHGAGVMTGAKFTQIQFGMTRQQVLDIAGAENCETGGSFGDSIHCRGHAAGAYYAYATFGFTSAAADAKVDSKSQEALLAPSAPTLTLAKFNQVTVGMTRAQVLATVGQGSCTTWSEYYPAYPSTAGVTLSLSCFDVDGYSSTGFYRGSAHLWFTDGVLQGKRQWDLVGEFGGWSHPQFEK

SEQ ID NO: 18 - protein, 633 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGWEKIRLRGGSGGSGGSGGSGGSGGSGAEAAAKEAAAKEAAAKEAAAKEAAAKEAAAKAGSGGSGGSGGPQGILGQGSGGSGGSGGSGAEAAAKEAAAKEAAAKEAAAKEAAAKEAAAKAGSGGSGGSGGSGGSGGSGGWEKIRLRGGSGGSHGAGVMTGAKFTQIQFGMTRQQVLDIAGAENCETGGSFGDSIHCRGHAAGDYYAYATFGFTSAAADAKVDSKSQMALLAPSAPTLTLAKFNQVTVGMTRAQVLATVGQGSCTTWSEYYPAYPSTAGVTLSLSCFDVDGYSSTGFYRGSAHLWFTDGVLQGKRQWDLVGEFGGWSHPQFEK

SEQ ID NO: 19 - protein, 633 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGWEKIRLRGGSGGSGGSGGSGGSGGSGAEAAAKEAAAKEAAAKEAAAKEAAAKEAAAKAGSGGSGGSGGPQGILGQGSGGSGGSGGSGAEAAAKEAAAKEAAAKEAAAKEAAAKEAAAKAGSGGSGGSGGSGGSGGSGGWEKIRLRGGSGGSHGAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGFYRGSAHLWFTDGVLQGKRQWDLVGEFGGWSHPQFEK

SEQ ID NO: 20 - protein, 633 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGWEKIRLRGGSGGSGGSGGSGGSGGSGAEAAAKEAAAKEAAAKEAAAKEAAAKEAAAKAGSGGSGGSGGPQGILGQGSGGSGGSGGSGAEAAAKEAAAKEAAAKEAAAKEAAAKEAAAKAGSGGSGGSGGSGGSGGSGGWEKIRLRGGSGGSHGAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGAYRGSAHLWFTDGVLQGKRQWDLVGEFGGWSHPQFEK

SEQ ID NO: 21 - protein, 629 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGELDRWEKIRLRPGGSGGSGGSGGSGGSGGSGAEAAAKEAAAKEAAAKEAAAKEAAAKEAAAKAGSGGSGGSGGSGGSGGSGAEAAAKEAAAKEAAAKEAAAKEAAAKEAAAKAGSGGSGGSGGSGGSGGSGGELDRWEKIRLRPGGSHGAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGFYRGSAHLWFTDGVLQGKRQWDLVGEFGGWSHPQFEK

SEQ ID NO: 22 - protein, 629 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGELDRWEKIRLRPGGSGGSGGSGGSGGSGGSGAEAAAKEAAAKEAAAKEAAAKEAAAKEAAAKAGSGGSGGSGGSGGSGGSGAEAAAKEAAAKEAAAKEAAAKEAAAKEAAAKAGSGGSGGSGGSGGSGGSGGELDRWEKIRLRPGGSHGAGVMTGAKFTQIQFGMTRQQVLDIAGAENCATGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGFYRGSAHLWFTDGVLQGKRQWDLVGEFGGWSHPQFEK

SEQ ID NO: 23 - protein, 629 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGELDRWEKIRLRPGGSGGSGGSGGSGGSGGSGAEAAAKEAAAKEAAAKEAAAKEAAAKEAAAKAGSGGSGGSGGSGGSGGSGAEAAAKEAAAKEAAAKEAAAKEAAAKEAAAKAGSGGSGGSGGSGGSGGSGGELDRWEKIRLRPGGSHGAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGFYRGSAHLWFTDGVLQGKRQWDLVGEFGGWSHPQFEK

SEQ ID NO: 24 - protein, 629 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGELDRWEKIRLRPGGSGGSGGSGGSGGSGGSGAEAAAKEAAAKEAAAKEAAAKEAAAKEAAAKAGSGGSGGSGGSGGSGGSGAEAAAKEAAAKEAAAKEAAAKEAAAKEAAAKAGSGGSGGSGGSGGSGGSGGELDRWEKIRLRPGGSHGAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGFYRGSAHLAFTDGVLQGKRQWDLVGEFGGWSHPQFEK

SEQ ID NO: 25 - protein, 629 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGELDRWEKIRLRPGGSGGSGGSGGSGGSGGSGAEAAAKEAAAKEAAAKEAAAKEAAAKEAAAKAGSGGSGGSGGSGGSGGSGAEAAAKEAAAKEAAAKEAAAKEAAAKEAAAKAGSGGSGGSGGSGGSGGSGGELDRWEKIRLRPGGSHGAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGFYRGSAHLWFTDGVLQGKAQWDLVGEFGGWSHPQFEK

SEQ ID NO: 26 - protein, 629 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGELDRWEKIRLRPGGSGGSGGSGGSGGSGGSGAEAAAKEAAAKEAAAKEAAAKEAAAKEAAAKAGSGGSGGSGGSGGSGGSGAEAAAKEAAAKEAAAKEAAAKEAAAKEAAAKAGSGGSGGSGGSGGSGGSGGELDRWEKIRLRPGGSHGAGVMTGAKFTQIQFGMTRQQVLDIAGAENCETGGSAGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGFYRGSAHLWFTDGVLQGKRQWDLVGEFGGWSHPQFEK

SEQ ID NO: 27 - protein, 629 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGELDRWEKIRLRPGGSGGSGGSGGSGGSGGSGAEAAAKEAAAKEAAAKEAAAKEAAAKEAAAKAGSGGSGGSGGSGGSGGSGAEAAAKEAAAKEAAAKEAAAKEAAAKEAAAKAGSGGSGGSGGSGGSGGSGGELDRWEKIRLRPGGSHGAGVMTGAKFTQIQFGMTRQQVLDIAGAENCETGGSFGDSIHCRGHAAGDYYAFATFGFTSAAADAKVDSKSQEKLLAPSAPTLTLAKFNQVTVGMTRAQVLATVGQGSCTTWSEYYPAYPSTAGVTLSLSCFDVDGYSSTGFYRGSAHLWFTDGVLQGKRQWDLVGEFGGWSHPQFEK

SEQ ID NO: 28 - protein, 629 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGELDRWEKIRLRPGGSGGSGGSGGSGGSGGSGAEAAAKEAAAKEAAAKEAAAKEAAAKEAAAKAGSGGSGGSGGSGGSGGSGAEAAAKEAAAKEAAAKEAAAKEAAAKEAAAKAGSGGSGGSGGSGGSGGSGGELDRWEKIRLRPGGSHGAGVMTGAKFTQIQFGMTRQQVLDIAGAENCETGGSFGDSIHCRGHAAGDYYAYATFGFTSAAADAKVDSKSQMKLLAPSAPTLTLAKFNQVTVGMTRAQVLATVGQGSCTTWSEYYPAYPSTAGVTLSLSCFDVDGYSSTGFYRGSAHLWFTDGVLQGKRQWDLVGEFGGWSHPQFEK

SEQ ID NO: 29 - protein, 629 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGELDRWEKIRLRPGGSGGSGGSGGSGGSGGSGAEAAAKEAAAKEAAAKEAAAKEAAAKEAAAKAGSGGSGGSGGSGGSGGSGAEAAAKEAAAKEAAAKEAAAKEAAAKEAAAKAGSGGSGGSGGSGGSGGSGGELDRWEKIRLRPGGSHGAGVMTGAKFTQIQFGMTRQQVLDIAGAENCETGGSFGDSIHCRGHAAGDYYAYATFGFTSAAADAKVDSKSQEALLAPSAPTLTLAKFNQVTVGMTRAQVLATVGQGSCTTWSEYYPAYPSTAGVTLSLSCFDVDGYSSTGFYRGSAHLWFTDGVLQGKRQWDLVGEFGGWSHPQFEK

SEQ ID NO: 30 - protein, 629 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGELDRWEKIRLRPGGSGGSGGSGGSGGSGGSGAEAAAKEAAAKEAAAKEAAAKEAAAKEAAAKAGSGGSGGSGGSGGSGGSGAEAAAKEAAAKEAAAKEAAAKEAAAKEAAAKAGSGGSGGSGGSGGSGGSGGELDRWEKIRLRPGGSHGAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTASEYYPAYPSTAGVTLSLSCFDVDGYSSTGFYRGSAHLWFTDGVLQGKRQWDLVGEFGGWSHPQFEK

SEQ ID NO: 31 - protein, 629 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGELDRWEKIRLRPGGSGGSGGSGGSGGSGGSGAEAAAKEAAAKEAAAKEAAAKEAAAKEAAAKAGSGGSGGSGGSGGSGGSGAEAAAKEAAAKEAAAKEAAAKEAAAKEAAAKAGSGGSGGSGGSGGSGGSGGELDRWEKIRLRPGGSHGAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGVYRGSAHLWFTDGVLQGKRQWDLVGEFGGWSHPQFEK

SEQ ID NO: 32 - protein, 629 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGELDRWEKIRLRPGGSGGSGGSGGSGGSGGSGAEAAAKEAAAKEAAAKEAAAKEAAAKEAAAKAGSGGSGGSGGSGGSGGSGAEAAAKEAAAKEAAAKEAAAKEAAAKEAAAKAGSGGSGGSGGSGGSGGSGGELDRWEKIRLRPGGSHGAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGYYRGSAHLWFTDGVLQGKRQWDLVGEFGGWSHPQFEK

SEQ ID NO: 33 - protein, 629 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGELDRWEKIRLRPGGSGGSGGSGGSGGSGGSGAEAAAKEAAAKEAAAKEAAAKEAAAKEAAAKAGSGGSGGSGGSGGSGGSGAEAAAKEAAAKEAAAKEAAAKEAAAKEAAAKAGSGGSGGSGGSGGSGGSGGELDRWEKIRLRPGGSHGAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGFYRGSAHLAFTDGVLQGKRQWDLVGEFGGWSHPQFEK

SEQ ID NO: 34 - protein, 629 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGELDRWEKIRLRPGGSGGSGGSGGSGGSGGSGAEAAAKEAAAKEAAAKEAAAKEAAAKEAAAKAGSGGSGGSGGSGGSGGSGAEAAAKEAAAKEAAAKEAAAKEAAAKEAAAKAGSGGSGGSGGSGGSGGSGGELDRWEKIRLRPGGSHGAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGFYRGSAHLFFTDGVLQGKRQWDLVGEFGGWSHPQFEK

SEQ ID NO: 35 - protein, 629 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGELDRWEKIRLRPGGSGGSGGSGGSGGSGGSGAEAAAKEAAAKEAAAKEAAAKEAAAKEAAAKAGSGGSGGSGGSGGSGGSGAEAAAKEAAAKEAAAKEAAAKEAAAKEAAAKAGSGGSGGSGGSGGSGGSGGELDRWEKIRLRPGGSHGAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGFYRGSAHLWFTDGVLQGKAQWDLVGEFGGWSHPQFEK

SEQ ID NO: 36 - protein, 629 aa, Unknown; identical to SEQ ID NO: 22
  synthetic construct

SEQ ID NO: 37 - protein, 629 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGELDRWEKIRLRPGGSGGSGGSGGSGGSGGSGAEAAAKEAAAKEAAAKEAAAKEAAAKEAAAKAGSGGSGGSGGSGGSGGSGAEAAAKEAAAKEAAAKEAAAKEAAAKEAAAKAGSGGSGGSGGSGGSGGSGGELDRWEKIRLRPGGSHGAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGAYRGSAHLWFTDGVLQGKRQWDLVGEFGGWSHPQFEK

SEQ ID NO: 38 - protein, 617 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGGGSGGSGGSGGSGGSGGSGAEAAAKEAAAKEAAAKEAAAKEAAAKEAAAKAGSGGSGGSGGSGGSGGSGAEAAAKEAAAKEAAAKEAAAKEAAAKEAAAKAGSGGSGGSGGSGGSGGSGGELDRWEKIRLRPGGSHGAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGAYRGSAHLWFTDGVLQGKRQWDLVGEFGGWSHPQFEK

SEQ ID NO: 39 - protein, 617 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGELDRWEKIRLRPGGSGGSGGSGGSGGSGGSGAEAAAKEAAAKEAAAKEAAAKEAAAKEAAAKAGSGGSGGSGGSGGSGGSGAEAAAKEAAAKEAAAKEAAAKEAAAKEAAAKAGSGGSGGSGGSGGSGGSGGGGSHGAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGAYRGSAHLWFTDGVLQGKRQWDLVGEFGGWSHPQFEK

SEQ ID NO: 40 - protein, 629 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGYPYDVPDYAGGSGGSGGGSGGSGAEAAAKEAAAKEAAAKEAAAKEAAAKEAAAKAGSGGSGGSGGPQGILGQGSGGSGGSGGSGAEAAAKEAAAKEAAAKEAAAKEAAAKEAAAKAGSGGSGGSGGSGGSGGSGGEYPYDVPDYAGGSHGAGVMTGAKFTQIQFGMTRQQVLDIAGAENCATGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGFYRGSAHLWFTDGVLQGKRQWDLVGEFGGWSHPQFEK

SEQ ID NO: 41 - protein, 629 aa, Unknown; identical to SEQ ID NO: 40
  synthetic construct

SEQ ID NO: 42 - protein, 629 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWTSGGEHKYSWKSGGSGGSGGGSGGSGAEAAAKEAAAKEAAAKEAAAKEAAAKEAAAKAGSGGSGGSGGPQGILGQGSGGSGGSGGSGAEAAAKEAAAKEAAAKEAAAKEAAAKEAAAKAGSGGSGGSGGSGGSGGSGGELEHKYSWKSGGSHGAGVMTGAKFTQIQFGMTRQQVLDIAGAENCATGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGFYRGSAHLWFTDGVLQGKRQWDLVGEFGGWSHPQFEK

SEQ ID NO: 43 - protein, 629 aa, Unknown; identical to SEQ ID NO: 42
  synthetic construct

SEQ ID NO: 44 - protein, 298 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 45 - protein, 310 aa, Unknown
  synthetic construct
MSIQHFRVALIPFFAAFCLPVFAHHHHHHLVPRGSHPETLVKVKDAEDQLGARVGYIELDLNSGKILESFRPEERFPMMSTFKVLLCGAVLSRIDAGQEQLGRRIHYSQNDLVDYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWEFGGWSHPQFEK

SEQ ID NO: 46 - protein, 206 aa, Unknown
  synthetic construct
MSIQHFRVALIPFFAAFCLPVFAHHHHHHLVPRGSSGGSHGAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGFYRGSAHLWFTDGVLQGKRQWDLV

SEQ ID NO: 47 - protein, 206 aa, Unknown
  synthetic construct
MSIQHFRVALIPFFAAFCLPVFAHHHHHHLVPRGSSGGSHGAGVMTGAKFTQIQFGMTRQQVLDIAGAENCATGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGFYRGSAHLWFTDGVLQGKRQWDLV

SEQ ID NO: 48 - protein, 206 aa, Unknown
  synthetic construct
MSIQHFRVALIPFFAAFCLPVFAHHHHHHLVPRGSSGGSHGAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGAYRGSAHLWFTDGVLQGKRQWDLV

SEQ ID NO: 49 - DNA, 34 bp, Unknown
  synthetic construct
cagaatgatctggtggaatatagtccggttacgg

SEQ ID NO: 50 - DNA, 26 bp, Unknown
  synthetic contruct
cggaaaactgcgcaaccggcggtagc

SEQ ID NO: 51 - DNA, 28 bp, Unknown
  synthetic construct
gcgaaaccggcggtagcgctggcgattc

SEQ ID NO: 52 - DNA, 30 bp, Unknown
  synthetic construct
gccacgcggccggtgcttattacgcctatg

SEQ ID NO: 53 - DNA, 36 bp, Unknown
  synthetic construct
ggtgattattacgcctttgcaacctttggtttcacc

SEQ ID NO: 54 - DNA, 42 bp, Unknown
  synthetic construct
gcggatgcaaaagtggatagtaaaagccagatgaaactgctg

SEQ ID NO: 55 - DNA, 30 bp, Unknown
  synthetic construct
ccaggaagccctgctggccccgagtgcacc

SEQ ID NO: 56 - DNA, 36 bp, Unknown
  synthetic construct
gggtagttgcaccacggccagcgaatattatccggc

SEQ ID NO: 57 - DNA, 35 bp, Unknown
  synthetic construct
gctatagctctaccggcgcttaccgtggttctgcg

SEQ ID NO: 58 - DNA, 33 bp, Unknown
  synthetic construct
ctatagctctaccggcgtttaccgtggttctgc

SEQ ID NO: 59 - DNA, 33 bp, Unknown
  synthetic construct
ctatagctctaccggctattaccgtggttctgc

SEQ ID NO: 60 - DNA, 31 bp, Unknown
  synthetic construct
cgtggttctgcggctctgtggttcacggatg

SEQ ID NO: 61 - DNA, 34 bp, Unknown
  synthetic construct
cgtggttctgcgcatctggctttcacggatggcg

SEQ ID NO: 62 - DNA, 33 bp, Unknown
  synthetic construct
ggttctgcgcatctgttcttcacggatggcgtg

SEQ ID NO: 63 - DNA, 32 bp, Unknown
  synthetic construct
cgtgctgcaaggtaaagcccagtgggatctgg

SEQ ID NO: 64 - DNA, 29 bp, Unknown
  synthetic construct
gccggattacgcgggcggttcccatggag

SEQ ID NO: 65 - DNA, 41 bp, Unknown
  synthetic construct
gcacatcgtacggataaccactagtccaatgtttaatcagg

SEQ ID NO: 66 - DNA, 44 bp, Unknown
  synthetic construct
tatccgtacgatgtgccggattacgcgggtggaagtgggggcag

SEQ ID NO: 67 - DNA, 43 bp, Unknown
  synthetic construct
cgcgtaatccggcacatcgtacggatactcacctccggaaccg

SEQ ID NO: 68 - DNA, 41 bp, Unknown
  synthetic construct
gaacataaatatagctggaaaagcggtggaagtgggggcag

SEQ ID NO: 69 - DNA, 45 bp, Unknown
  synthetic construct
ccgcttttccagctatatttatgttctaactcacctccggaaccg

SEQ ID NO: 70 - DNA, 40 bp, Unknown
  synthetic construct
gaacataaatatagctggaaaagcggcggttcccatggag

SEQ ID NO: 71 - DNA, 49 bp, Unknown
  synthetic construct
gcttttccagctatatttatgttcgccaccactagtccaatgtttaatc